(19) United States
(12) Patent Application Publication
(10) Pub. No.: US 2023/0332146 A1
(43) Pub. Date: Oct. 19, 2023

(54) DIFFERENTIAL KNOCKOUT OF A HETEROZYGOUS ALLELE OF SAMD9

(71) Applicants: Rafi Emmanuel, Ramla (IL); Joseph Dicken, Rehovot (IL); Michal Golan Mashiach, Ness Ziona (IL)

(72) Inventors: Rafi Emmanuel, Ramla (IL); Joseph Dicken, Rehovot (IL); Michal Golan Mashiach, Ness Ziona (IL)

(73) Assignee: EmendoBio Inc., Wilmington, DE (US)

(21) Appl. No.: 17/927,671

(22) PCT Filed: May 28, 2021
(86) PCT No.: PCT/US2021/034873

§ 371 Date: Nov. 23, 2022

Related U.S. Application Data
(60) Provisional application No. 63/031,175, filed on May 28, 2020.

(51) Int. Cl. C12N 15/113; C12N 15/90

(52) U.S. Cl. CPC C12N 15/113; C12N 15/907; C12N 2310/20; C12N 2320/34

(57) ABSTRACT

RNA molecules comprising a guide sequence portion having 17-50 contiguous nucleotides containing nucleotides in the sequence set forth in any one of SEQ ID NOs: 1-20640 and compositions, methods, and uses thereof.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “210528_91330-A-PCT_Sequence_Listing_AWG.txt”, which is 4,392 kilobytes in size, and which was created on May 27, 2021 in the IBM-PC machine format, having an operating system compatibility with MS-Windows, which is contained in the text file filed May 28, 2021 as part of this application.

### BACKGROUND OF INVENTION

There are several classes of DNA variation in the human genome, including insertions and deletions, differences in the copy number of repeated sequences, and single nucleotide polymorphisms (SNPs). A SNP is a DNA sequence variation occurring when a single nucleotide (adenine (A), thymine (T), cytosine (C), or guanine (G)) in the genome differs between human subjects or paired chromosomes in an individual. Over the years, the different types of DNA variations have been the focus of the research community either as markers in studies to pinpoint traits or disease causation or as potential causes of genetic disorders.

A genetic disorder is caused by one or more abnormalities in the genome. Genetic disorders may be regarded as either “dominant” or “recessive.” Recessive genetic disorders are those which require two copies (i.e., two alleles) of the abnormal/defective gene to be present. In contrast, a dominant genetic disorder involves a gene or genes which exhibit(s) dominance over a normal (functional/healthy) gene or genes. As such, in dominant genetic disorders only a single copy (i.e., allele) of an abnormal gene is required to cause or contribute to the symptoms of a particular genetic disorder. Such mutations include, for example, gain-of-function mutations in which the altered gene product possesses a new molecular function or a new pattern of gene expression. Other examples include dominant negative mutations, which have a gene product that acts antagonistically to the wild-type allele.

### MIRAGE SYNDROME

Sterile alpha motif domain containing 9 (SAMD9) is a protein encoding gene that is involved in control of cell proliferation and functions as a tumor suppressor in some cancers (Davidsson et al., 2018). Several dominant negative mutations of the SAMD9 gene were shown to be associated with MIRAGE syndrome, which is a rare disorder that manifests as a form of syndromic adrenal hypoplasia, characterized by myelodysplasia, infection, restriction of growth, adrenal hypoplasia, genital phenotypes, and enteropathy (Narumi et al., 2016).

### SUMMARY OF THE INVENTION

Disclosed is an approach for knocking out the expression of a dominant-mutated allele by disrupting the dominant-mutated allele or degrading the resulting mRNA.

The present disclosure provides a method for utilizing at least one naturally occurring nucleotide difference or polymorphism (e.g., single nucleotide polymorphism (SNP)) for distinguishing/discriminating between two alleles of a gene, one allele bearing a mutation such that it encodes a mutated protein causing a disease phenotype (“mutated allele”) and a particular sequence in a SNP position (REF/SNP), and the other allele encoding for a functional protein (“functional allele”). In some embodiments, the SNP position is utilized for distinguishing/discriminating between two alleles of a gene bearing one or more disease associated mutations, such as to target one of the alleles bearing both the particular sequence in the SNP position (SNP/REF) and a disease associated mutation. In some embodiments, the disease-associated mutation is targeted. In some embodiments, the method further comprises the step of knocking out expression of the mutated protein and allowing expression of the functional protein.

The present disclosure also provides a method for modifying in a cell a mutant allele of the sterile alpha motif domain containing 9 (SAMD9) gene having a mutation associated with MIRAGE syndrome, the method comprising introducing to the cell a composition comprising:
a CRISPR nuclease or a sequence encoding the CRISPR nuclease; and
a first RNA molecule comprising a guide sequence portion having 17-50 nucleotides or a nucleotide sequence encoding the same,
wherein a complex of the CRISPR nuclease and the first RNA molecule affects a double strand break in the mutant allele of the SAMD9 gene.

According to embodiments of the present invention, there is provided a first RNA molecule comprising a guide sequence portion having 17-50 contiguous nucleotides containing nucleotides in the sequence set forth in any one of SEQ ID Nos: 1-20640.

According to some embodiments of the present invention, there is provided a composition comprising an RNA molecule comprising a guide sequence portion having 17-50 contiguous nucleotides containing nucleotides in the sequence set forth in any one of SEQ ID NOs: 1-20640 and a CRISPR nuclease.

According to some embodiments of the present invention, there is provided a method for inactivating a mutant SAMD9 allele in a cell, the method comprising delivering to the cell a composition comprising an RNA molecule comprising a guide sequence portion having 17-50 contiguous nucleotides containing nucleotides in the sequence set forth in any one of SEQ ID NOs: 1-20640 and a CRISPR nuclease. In some embodiments, the cell is a stem cell. In some embodiments, the stem cell is a hematopoietic stem/progenitor cell (HSC). In some embodiments, the delivering to the cell is performed in vitro, ex vivo, or in vivo. In some embodiments, the method is performed ex vivo and the cell is provided/explanted from an individual patient. In some embodiments, the method further comprises the step of introducing the resulting cell, with the modified/knocked out mutant SAMD9 allele, into the individual patient (e.g. autologous transplantation).

According to some embodiments of the present invention, there is provided a method for treating MIRAGE syndrome, the method comprising delivering to a cell of a subject having MIRAGE syndrome a composition comprising an RNA molecule comprising a guide sequence portion having 17-50 contiguous nucleotides containing nucleotides in the sequence set forth in any one of SEQ ID NOs: 1-20640 and a CRISPR nuclease.

According to some embodiments of the present invention, there is provided use of a composition comprising an RNA molecule comprising a guide sequence portion having 17-50 contiguous nucleotides containing nucleotides in the sequence set forth in any one of SEQ ID NOs: 1-20640 and a CRISPR nuclease for inactivating a mutant SAMD9 allele in a cell, comprising delivering to the cell the composition comprising an RNA molecule comprising a guide sequence portion having 17-50 contiguous nucleotides containing nucleotides in the sequence set forth in any one of SEQ ID NOs: 1-20640 and a CRISPR nuclease.

According to embodiments of the present invention, there is provided a medicament comprising an RNA molecule comprising a guide sequence portion having 17-50 contiguous nucleotides containing nucleotides in the sequence set forth in any one of SEQ ID NOs: 1-20640 and a CRISPR nuclease for use in inactivating a mutant SAMD9 allele in a cell, wherein the medicament is administered by delivering to the cell the composition comprising an RNA molecule comprising a guide sequence portion having 17-50 contiguous nucleotides containing nucleotides in the sequence set forth in any one of SEQ ID NOs: 1-20640 and a CRISPR nuclease.

According to some embodiments of the present invention, there is provided use of a composition comprising an RNA molecule comprising a guide sequence portion having 17-50 contiguous nucleotides containing nucleotides in the sequence set forth in any one of SEQ ID NOs: 1-20640 and a CRISPR nuclease for treating ameliorating or preventing MIRAGE syndrome, comprising delivering to a cell of a subject having or at risk of having MIRAGE syndrome the composition of comprising an RNA molecule comprising a guide sequence portion having 17-50 contiguous nucleotides containing nucleotides in the sequence set forth in any one of SEQ ID NOs: 1-20640 and a CRISPR nuclease. In some embodiments, the method is performed ex vivo and the cell is provided/explanted from the subject. In some embodiments, the method further comprises the step of introducing the resulting cell, with the modified/knocked out mutant SAMD9 allele, into the subject (e.g. autologous transplantation).

According to some embodiments of the present invention, there is provided a medicament comprising the composition comprising an RNA molecule comprising a guide sequence portion having 17-50 contiguous nucleotides containing nucleotides in the sequence set forth in any one of SEQ ID NOs: 1-20640 and a CRISPR nuclease for use in treating ameliorating or preventing MIRAGE syndrome, wherein the medicament is administered by delivering to a cell of a subject having or at risk of having MIRAGE syndrome the composition comprising an RNA molecule comprising a guide sequence portion having 17-50 contiguous nucleotides containing nucleotides in the sequence set forth in any one of SEQ ID NOs: 1-20640 and a CRISPR nuclease.

According to some embodiments of the present invention, there is provided a kit for inactivating a mutant SAMD9 allele in a cell, comprising an RNA molecule comprising a guide sequence portion having 17-50 contiguous nucleotides containing nucleotides in the sequence set forth in any one of SEQ ID NOs: 1-20640, a CRISPR nuclease, and/or a tracrRNA molecule; and instructions for delivering the RNA molecule; CRISPR nuclease, and/or the tracrRNA to the cell.

According to some embodiments of the present invention, there is provided a kit for treating MIRAGE syndrome in a subject, comprising an RNA molecule comprising a guide sequence portion having 17-50 contiguous nucleotides containing nucleotides in the sequence set forth in any one of SEQ ID NOs: 1-20640, a CRISPR nuclease, and/or a tracrRNA molecule; and instructions for delivering the RNA molecule; CRISPR nuclease, and/or the tracrRNA to a cell of a subject having or at risk of having MIRAGE syndrome.

### BRIEF DESCRIPTION OF THE DRAWINGS

FIG. 1: Guide activity of RNA guide molecules targeting SAMD9 in HeLa cells. An RNA guide molecule was co-transfected with SpCas9 to determine their on-target activity. Cells were harvested 72 hours post DNA transfection, genomic was DNA extracted, and the region of the mutation was amplified and analyzed by next-generation sequencing (NGS). The graph represents the % editing ± standard deviation (STDV) of three (3) independent transfections.

FIG. 2: Guide activity of novel OMNI CRISPR nucleases with RNA guide molecules targeting SAMD9 in HeLa cells. An RNA guide molecule was-co-transfected with each OMNI CRISPR nuclease to determine its on-target activity. Cells were harvested 72 hours post DNA transfection, genomic DNA was extracted, and the region of the mutation was amplified and analyzed by NGS. Transfection efficiency is measured by mCherry fluorescence. The graph represents the % editing ± STDV of three (3) independent transfections.

FIG. 3: Guide activity of RNA guide molecules targeting SAMD9 in donor-derived HSCs. RNPs complexed with SpCas9 and an RNA guide molecule were electroporated into HSCs to determine their activity. Cells were harvested 72 hours post RNP electroporation, genomic was DNA extracted, and the region of the mutation was amplified and analyzed by NGS. The graph represents the % editing ± STDV of two (2) independent electroporations. The table below the graph indicates the genotype of the HSCs for each SNP.

FIGS. 4A-4B: Effect of editing on protein levels of SAMD9 in U2OS cells. RNPs complexed with SpCas9 and a RNA guide molecule were electroporated into U2OSs. Cells were harvested six days post RNP electroporation, cells were lysed and analyzed by western blot (WB) assay (FIG. 4A). Relative protein levels to control (NT1 - control) were quantified for each sample (FIG. 4B).

FIGS. 5A-5D: Knockout strategies utilizing guides targeting SAMD9 - Schematic representation of example SAMD9 editing strategies. The SAMD9 protein-encoding exon is represented as a white box, the UTR regions are represented as dotted boxes, introns are represented as black lines, a nuclease is represented by scissors accompanied by an arrow that indicates the target site, a SNP is represented by a black star, a polyadenylation signal is represented by a black triangle, and a template encoding a splice acceptor is represented by a striped box. FIG. 5A: Strategy 1 - Creation of a frameshift generated by the use of a single discriminatory guide RNA molecule targeting a SNP within a mutated allele of a SAMD9 coding exon. FIG. 5B: Strategy 2 - Excision of the mutated SAMD9 coding exon using a first discriminatory guide RNA molecule and a second non-discriminatory guide RNA molecule. In the depicted schematic, the first discriminatory guide targets a SNP in Intron 2 and the second non-discriminatory guide RNA molecule targets the 3′UTR. In an alternative example of exon excision, the first discriminatory guide targets a SNP in the 3′UTR and the second non-discriminatory guide RNA molecule targets Intron 2. FIG. 5C: Strategy 3 - Excision of the polyadenylation signal of a mutated SAMD9 allele using a first discriminatory guide RNA molecule and a second non-discriminatory guide RNA molecule. FIG. 5D: Strategy 4 - Introduction of a splice acceptor site upstream of the coding exon of a mutated SAMD9 allele upon targeting a CRISPR nuclease to Intron 2 of the mutated allele using a discriminatory guide RNA molecule.

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length, preferably between about 100 and 1,000 nucleotides in length, more preferably between about 200 and 500 nucleotides in length. A nucleic acid template may be a single stranded nucleic acid, a double stranded nucleic acid. In some embodiments, the nucleic acid template comprises a nucleotide sequence, e.g., of one or more nucleotides, that corresponds to wild type sequence of the target nucleic acid, e.g., of the target position. In some embodiments, the nucleic acid template comprises a nucleotide sequence, e.g., of one or more ribonucleotides, that corresponds to wild type sequence of the target nucleic acid, e.g., of the target position. In some embodiments, the nucleic acid template comprises modified nucleotides.

In some embodiments of the present invention, a DNA nuclease is utilized to affect a DNA break at a target site to induce cellular repair mechanisms, for example, but not limited to, non-homologous end-joining (NHEJ). During classical NHEJ, two ends of a double-strand break (DSB) site are ligated together in a fast but also inaccurate manner (i.e. frequently resulting in mutation of the DNA at the cleavage site in the form of small insertion or deletions) whereas during HDR, an intact homologous DNA donor is used to replace the DNA surrounding the cleavage site in an accurate manner. HDR can also mediate the precise insertion of exogenous DNA at the break site. Accordingly, the term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

Insertion of an exogenous sequence (also called a “donor sequence,” donor template,” “donor molecule” or “donor”), for example, for insertion of a splice site to knockout expression of a gene, can also be carried out. For example, donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest. A donor molecule may be any length, for example ranging from several bases e.g. 10-20 bases to multiple kilobases in length.

The donor polynucleotide can be DNA or RNA, single-stranded and/or double-stranded and can be introduced into a cell in linear or circular form. See, e.g., U.S. Pat. Publication Nos. 2010/0047805; 2011/0281361; 2011/0207221; and 2019/0330620. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) and Nehls et al. (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Accordingly, embodiments of the present invention may use a donor template for HDR that is DNA or RNA, single-stranded and/or double-stranded and can be introduced into a cell in linear or circular form.

A donor sequence may be an oligonucleotide and be used for targeted alteration of an endogenous sequence. The oligonucleotide may be introduced to the cell on a vector, may be electroporated into the cell, or may be introduced via other methods known in the art. The oligonucleotide can be used to insert a sequence with a desired purpose into an endogenous locus (e.g. a splice acceptor sequence to prevent expression of a coding exon).

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor molecule may be inserted into an endogenous gene such that all, some or none of the endogenous gene is expressed.

Furthermore, although not required for expression, an exogenous sequence of a template may also include transcriptional or translational regulatory sequences, for example, splice donor or splice acceptor sites.

As used herein, the term “modified cells” refers to cells in which a double strand break is affected by a complex of an RNA molecule and the CRISPR nuclease as a result of hybridization with the target sequence, i.e. on-target hybridization. The term “modified cells” may further encompass cells in which a repair or correction of a mutation was affected following the double strand break.

This invention provides a modified cell or cells obtained by use of any of the methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cell (HSC), or any cell suitable for an allogenic cell transplant or autologous cell transplant.

This invention also provides a composition comprising these modified cells and a pharmaceutically acceptable carrier. Also provided is an in vitro or ex vivo method of preparing this, comprising mixing the cells with the pharmaceutically acceptable carrier.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR molecule, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. The entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the DNA molecule having the guide sequence portion is present contemporaneously with the CRISPR molecule the RNA molecule is capable of targeting the CRISPR nuclease to the specific target DNA sequence. Each possibility represents a separate embodiment. An RNA molecule can be custom designed to target any desired sequence. Accordingly, a molecule comprising a “guide sequence portion” is a type of targeting molecule. In some embodiments, the guide sequence portion comprises a sequence that is the same as, or differs by no more than 1, 2, 3, 4, or 5 nucleotides from, a guide sequence portion described herein, e.g., a guide sequence set forth in any of SEQ ID NOs: 1-20640. Each possibility represents a separate embodiment. In some of these embodiments, the guide sequence portion is fully complementary to the target sequence, and comprises a sequence that is the same as a sequence set forth in any of SEQ ID NOs: 1-20640. Throughout this application, the terms “guide molecule,” “RNA guide molecule,” “guide RNA molecule,” and “gRNA molecule” are synonymous with a molecule comprising a guide sequence portion.

The term “non-discriminatory” as used herein refers to a guide sequence portion of an RNA molecule that targets a specific DNA sequence that is common both a mutant and functional allele of a gene.

In embodiments of the present invention, an RNA molecule comprises a guide sequence portion having 17-50 contiguous nucleotides containing nucleotides in the sequence set forth in any one of SEQ ID NOs: 1-20640.

The RNA molecule and or the guide sequence portion of the RNA molecule may contain modified nucleotides. Exemplary modifications to nucleotides / polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. 8,278,036, PCT International Publication No. WO/2015/006747, and Weissman and Kariko (2015), hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 25 nucleotides in length and contain 20-22 contiguous nucleotides in the sequence set forth in any one of SEQ ID NOs: 1-20640. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-20640. For example, a guide sequence portion having 17 nucleotides in the sequence of 17 contiguous nucleotides set forth in SEQ ID NO: 20641 may consist of any one of the following nucleotide sequences (nucleotides excluded from the contiguous sequence are marked in strike-through):

[image]

In embodiments of the present invention, the guide sequence portion may be greater than 20 nucleotides in length. For example, in embodiments of the present invention the guide sequence portion may be 21, 22, 23, 24 or 25 nucleotides in length. In such embodiments the guide sequence portion comprises 17-50 nucleotides containing the sequence of 20, 21 or 22 contiguous nucleotides set forth in any one of SEQ ID NOs: 1-20640 and additional nucleotides fully complimentary to a nucleotide or sequence of nucleotides adjacent to the 3′ end of the target sequence, 5′ end of the target sequence, or both.

In embodiments of the present invention a CRISPR nuclease and an RNA molecule comprising a guide sequence portion form a CRISPR complex that binds to a target DNA sequence to effect cleavage of the target DNA sequence. CRISPR nucleases, e.g. Cpfl, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule. A guide sequence portion, which comprises a nucleotide sequence that is capable of hybridizing to a specific target DNA sequence, and a sequence portion that participates in CRIPSR nuclease binding, e.g. a tracrRNA sequence portion, can be located on the same RNA molecule. Alternatively, a guide sequence portion may be located on one RNA molecule and a sequence portion that participates in CRIPSR nuclease binding, e.g. a tracrRNA portion, may located on a separate RNA molecule. A single RNA molecule comprising a guide sequence portion (e.g. a DNA-targeting RNA sequence) and at least one CRISPR protein-binding RNA sequence portion (e.g. a tracrRNA sequence portion), can form a complex with a CRISPR nuclease and serve as the DNA-targeting molecule. In some embodiments, a first RNA molecule comprising a DNA-targeting RNA portion, which includes a guide sequence portion, and a second RNA molecule comprising a CRISPR protein-binding RNA sequence interact by base pairing to form an RNA complex that targets the CRISPR nuclease to a DNA target site or, alternatively, are fused together to form an RNA molecule that complexes with the CRISPR nuclease and targets the CRISPR nuclease to a DNA target site.

In embodiments of the present invention, a RNA molecule comprising a guide sequence portion may further comprise the sequence of a tracrRNA molecule. Such embodiments may be designed as a synthetic fusion of the guide portion of the RNA molecule and the trans-activating crRNA (tracrRNA). (See Jinek et al., 2012). In such an embodiment, the RNA molecule is a single guide RNA (sgRNA) molecule. Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

As used herein, the term “HSC” refers to both hematopoietic stem cells and hematopoietic stem progenitor cells. Non-limiting examples of stem cells include bone marrow cells, myeloid progenitor cells, a multipotent progenitor cells, a lineage restricted progenitor cells.

As used herein, “progenitor cell” refers to a lineage cell that is derived from stem cell and retains mitotic capacity and multipotency (e.g., can differentiate or develop into more than one but not all types of mature lineage of cell). As used herein “hematopoiesis” or “hemopoiesis” refers to the formation and development of various types of blood cells (e.g., red blood cells, megakaryocytes, myeloid cells (e.g., monocytes, macrophages and neutrophil), and lymphocytes) and other formed elements in the body (e.g., in the bone marrow).

The term “single nucleotide polymorphism (SNP) position”, as used herein, refers to a position in which a single nucleotide DNA sequence variation occurs between members of a species, or between paired chromosomes in an individual. In the case that a SNP position exists at paired chromosomes in an individual, a SNP on one of the chromosomes is a “heterozygous SNP.” The term SNP position refers to the particular nucleic acid position where a specific variation occurs and encompasses both a sequence including the variation from the most frequently occurring base at the particular nucleic acid position (also referred to as “SNP” or alternative “ALT”) and a sequence including the most frequently occurring base at the particular nucleic acid position (also referred to as reference, or “REF”). Accordingly, the sequence of a SNP position may reflect a SNP (i.e. an alternative sequence variant relative to a consensus reference sequence within a population), or the reference sequence itself.

According to embodiments of the present invention, there is provided a method for modifying in a cell a mutant allele of the sterile alpha motif domain containing 9 (SAMD9) gene having a mutation associated with MIRAGE syndrome, the method comprising introducing to the cell a composition comprising:
at least one CRISPR nuclease or a sequence encoding a CRISPR nuclease; and
a first RNA molecule comprising a guide sequence portion having 17-50 nucleotides or a nucleotide sequence encoding the same,
wherein a complex of the CRISPR nuclease and the first RNA molecule affects a double strand break in the mutant allele of the SAMD9 gene.

In some embodiments, the first RNA molecule targets the CRISPR nuclease to the mutation associated with MIRAGE syndrome.

In some embodiments, the mutation associated with MIRAGE syndrome is any one of 7:93101391_C_G, 7:93101435_G_A, 7:93101512_C_T, 7:93101540_C_A, 7:93101615_T_C, 7:93101761_AGTT_A, 7:93101900_G_A, 7:93102220_C_T, 7:93102221_G_A, 7:93102242_G_T, 7:93102259_G_A, 7:93102368_T_C, 7:93102514_G_A, 7:93102593_G_A, 7:93102692_C_G, 7:93102717_G_T, 7:93103150_AT_GA, 7:93103151_T_C, 7:93103153_C_T, 7:93103178_C_T, 7:93103253_C_A, 7:93103393_C_T, 7:93103588_C_CT, 7:93103598_T_A, 7:93103628_G_A, 7:93103679_C_T, 7:93103765_G_A, 7:93103772_G_T, 7:93103793_C_T, 7:93103851_G_T, 7:93103900_A_G, 7:93103916_T_C, 7:93103933_G_T, 7:93103938_GT_G, 7:93104044_C_T, 7:93104176_A_G, 7:93104296_ATT_A, 7:93104449_T_A, 7:93104694_TAC_GCA, 7:93104695_AC_GA, 7:93104722_C_T, 7:93105043_A_G, 7:93105103_A_G, 7:93105122_G_A, 7:93105295_A_G, 7:93105436_C_T, 7:93105485_T_G, 7:93105638_G_A, 7:93105781_C_T, 7:93105965_CT_C, 7:93106002_GT_G, 7:93106003_T_A, and 7:93106096_A_G.

In some embodiments, the guide sequence portion of the first RNA molecule comprises 17-50 contiguous nucleotides containing nucleotides in the sequence set forth in any one of SEQ ID NOs: 1-20640 that targets a mutation associated with MIRAGE syndrome.

In some embodiments, the method further comprises introduction of a donor molecule for alteration of the SAMD9 mutant allele. In some embodiments, the donor molecule encodes a synthetic splice site (e.g. a splice acceptor or splice donor site) for insertion into the SAMD9 mutant allele.

In some embodiments, the first RNA molecule targets the CRISPR nuclease to a SNP position of the mutant allele.

In some embodiments, the SNP position is any one of rs201891812, rs73216083, rs542330750, rs7798225, rs10953089, rs10953090, rs10277338, rs71562815, rs12333976, rs3839837, rs10279499, rs78564070, rs6965201, rs6969691, rs2374627, rs12668221, rs28673511, rs10229795, rs7785869, rs67885698, rs6957832 and rs12704664.

In some embodiments, the guide sequence portion of the first RNA molecule comprises 17-50 contiguous nucleotides containing nucleotides in the sequence set forth in any one of SEQ ID NOs: 1-20640 that targets a SNP position of the mutant allele.

In some embodiments, the SNP position is in an exon of the SAMD9 mutant allele.

In some embodiments, the SNP position contains a heterozygous SNP.

In some embodiments, the method further comprises introducing to the cell a second RNA molecule comprising a guide sequence portion having 17-50 nucleotides or a nucleotide sequence encoding the same, wherein a complex of the second RNA molecule and a CRISPR nuclease affects a second double strand break in the SAMD9 gene.

In some embodiments, the guide sequence portion of the second RNA molecule comprises 17-50 contiguous nucleotides containing nucleotides in the sequence set forth in any one of SEQ ID NOs: 1-20640 other than the sequence of the first RNA molecule.

In some embodiments, the second RNA molecule comprises a non-discriminatory guide portion that targets both functional and mutated SAMD9 alleles.

In some embodiments, the second RNA molecule comprises a non-discriminatory guide portion that targets any one of a SAMD9 untranslated region (UTR), an intergenic region upstream of SAMD9, an intergenic region downstream of SAMD9, or Intron 2 of SAMD9.

In some embodiments, the second RNA molecule comprises a non-discriminatory guide portion that targets a sequence that is located within a genomic range selected from any one of 7:93100828 to 7:93101327, 7:93099521 to 7:93100020, 7:93099413 to 7:93099913, or 7:93106106 to 7:93106605.

In some embodiments, the second RNA molecule comprises a non-discriminatory guide portion that targets a sequence that is located up to 500 base pairs from the sequence targeted by the first RNA molecule.

In some embodiments, a portion of an exon is excised from the mutant allele of the SAMD9 gene.

In some embodiments, the first RNA molecule targets a SNP position in the 3′ UTR of the mutated allele, and the second RNA molecule comprises a non-discriminatory guide portion that targets downstream of a polyadenylation signal sequence that is common to both a functional allele and the mutant allele of the SAMD9 gene.

In some embodiments, the first RNA molecule targets a SNP position downstream of a polyadenylation signal of the mutated allele, and the second RNA molecule comprises a non-discriminatory guide portion that targets a sequence upstream of a polyadenylation signal that is common to both a functional allele and the mutant allele of the SAMD9 gene.

In some embodiments, the polyadenylation signal is excised from the mutant allele of the SAMD9 gene.

According to embodiments of the present invention, there is provided a modified cell obtained by the method of any one of the embodiments presented herein.

According to embodiments of the present invention, there is provided a first RNA molecule comprising a guide sequence portion having 17-50 contiguous nucleotides containing nucleotides in the sequence set forth in any one of SEQ ID NOs: 1-20640.

According to embodiments of the present invention, there is provided a composition comprising the first RNA molecule and at least one CRISPR nuclease.

In some embodiments, the composition further comprises a second RNA molecule comprising a guide sequence portion having 17-50 contiguous nucleotides, wherein the second RNA molecule targets a SAMD9 allele, and wherein the guide sequence portion of the second RNA molecule is a different sequence from the sequence of the guide sequence portion of the first RNA molecule.

In some embodiments, the guide sequence portion of the second RNA molecule comprises 17-50 contiguous nucleotides containing nucleotides in the sequence set forth in any one of SEQ ID NOs: 1-20640 other than the sequence of the first RNA molecule.

According to embodiments of the present invention, there is provided a method for inactivating a mutant SAMD9 allele in a cell, the method comprising delivering to the cell the composition of any one of the embodiments presented herein.

According to embodiments of the present invention, there is provided a method for treating MIRAGE syndrome, the method comprising delivering to a cell of a subject having MIRAGE syndrome the composition of any one of the embodiments presented herein.

According to embodiments of the present invention, there is provided use of any one of the compositions presented herein for inactivating a mutant SAMD9 allele in a cell, comprising delivering to the cell the composition of any one of the embodiments presented herein.

According to embodiments of the present invention, there is provided a medicament comprising the composition of any one of the embodiments presented herein for use in inactivating a mutant SAMD9 allele in a cell, wherein the medicament is administered by delivering to the cell the composition of any one of the embodiments presented herein.

According to embodiments of the present invention, there is provided use of the composition of any one of the embodiments presented herein for treating ameliorating or preventing MIRAGE syndrome, comprising delivering to a cell of a subject having or at risk of having MIRAGE syndrome the composition of any one of the embodiments presented herein.

According to embodiments of the present invention, there is provided a medicament comprising the composition of any one of the embodiments presented herein for use in treating ameliorating or preventing MIRAGE syndrome, wherein the medicament is administered by delivering to a cell of a subject having or at risk of having MIRAGE syndrome the composition of any one of the embodiments presented herein.

According to embodiments of the present invention, there is provided a kit for inactivating a mutant SAMD9 allele in a cell, comprising an RNA molecule of any one of the embodiments presented herein, a CRISPR nuclease, and/or a tracrRNA molecule; and instructions for delivering the RNA molecule; CRISPR nuclease, and/or the tracrRNA to the cell.

According to embodiments of the present invention, there is provided a kit for treating MIRAGE syndrome in a subject, comprising an RNA molecule of any one of the embodiments presented herein, a CRISPR nuclease, and/or a tracrRNA molecule; and instructions for delivering the RNA molecule; CRISPR nuclease, and/or the tracrRNA to a cell of a subject having or at risk of having MIRAGE syndrome.

According to embodiments of the present invention, there is provided a gene editing composition comprising an RNA molecule comprising a guide sequence portion having 17-50 contiguous nucleotides containing nucleotides in the sequence set forth in any one of SEQ ID NOs: 1-20640. In some embodiments, the RNA molecule further comprises a portion having a sequence which binds to a CRISPR nuclease. In some embodiments, the sequence which binds to a CRISPR nuclease is a tracrRNA sequence.

In some embodiments, the RNA molecule further comprises a portion having a tracr mate sequence.

In some embodiments, the RNA molecule may further comprise one or more linker portions.

According to embodiments of the present invention, an RNA molecule may be up to 1000, 900, 800, 700, 600, 500, 450, 400, 350, 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 100 up to 500 nucleotides in length, 100 up to 400 nucleotides in length, 200 up to 300 nucleotides in length, 100 to 200 nucleotides in length, or 150 up to 250 nucleotides in length. Each possibility represents a separate embodiment.

According to some embodiments of the present invention, the composition further comprises a tracrRNA molecule.

The present disclosure provides a method for utilizing at least one naturally occurring nucleotide difference or polymorphism (e.g., single nucleotide polymorphism (SNP)) for distinguishing/discriminating between two alleles of a gene, one allele bearing a mutation such that it encodes a mutated protein causing a disease phenotype (“mutated allele”) and a particular sequence in a SNP position (SNP/REF), and the other allele encoding for a functional protein (“functional allele”). The method further comprises the step of knocking out expression of the mutated protein and allowing expression of the functional protein. In some embodiments, the method is for treating, ameliorating, or preventing a dominant negative genetic disorder.

According to some embodiments of the present invention, there is provided a method for inactivating a mutant SAMD9 allele in a cell, the method comprising delivering to the cell a composition comprising an RNA molecule comprising a guide sequence portion having 17-50 contiguous nucleotides containing nucleotides in the sequence set forth in any one of SEQ ID NOs: 1-20640 and a CRISPR nuclease.

According to some embodiments of the present invention, there is provided a method for treating MIRAGE syndrome, the method comprising delivering to a cell of a subject having MIRAGE syndrome a composition comprising an RNA molecule comprising a guide sequence portion having 17-50 contiguous nucleotides containing nucleotides in the sequence set forth in any one of SEQ ID NOs: 1-20640 and a CRISPR nuclease.

According to embodiments of the present invention, the composition comprises a second RNA molecule comprising a guide sequence portion having 17-50 contiguous nucleotides containing nucleotides in the sequence set forth in any one of SEQ ID NOs: 1-20640. In some embodiments, the 17-50 nucleotides of the guide sequence portion of the second RNA molecule are in a different sequence from the sequence of the guide sequence portion of the first RNA molecule.

According to embodiments of the present invention, at least one CRISPR nuclease and the RNA molecule or RNA molecules are delivered to the subject and/or cells substantially at the same time or at different times.

In some embodiments, a tracrRNA molecule is delivered to the subject and/or cells substantially at the same time or at different times as the CRISPR nuclease and RNA molecule or RNA molecules.

According to embodiments of the present invention, the first RNA molecule targets a SNP or disease-causing mutation in the exon or promoter of a mutated allele, and the second RNA molecule targets a SNP in an exon of the mutated allele, a SNP in an intron, or a sequence present in both the mutated or functional allele.

According to embodiments of the present invention, the first RNA molecule or the first and the second RNA molecules target a SNP in the promoter region, the start codon, or an untranslated region (UTR) of a mutated allele.

According to embodiments of the present invention, the first RNA molecule or the first and the second RNA molecules targets at least a portion of the promoter and/or the start codon and/or a portion of a UTR of a mutated allele.

According to embodiments of the present invention, the first RNA molecule targets a portion of the promoter, a first SNP in the promoter, or a SNP upstream to the promoter of a mutated allele and the second RNA molecule is targets a second SNP, which is downstream of the first SNP, and is in the promoter, in a UTR, or in an intron or in an exon of a mutated allele.

According to embodiments of the present invention, the first RNA molecule targets a SNP in the promoter, upstream of the promoter, or a UTR of a mutated allele and the second RNA molecule is designed to target a sequence which is present in an intron of both the mutated allele and the functional allele.

According to embodiments of the present invention, the first RNA molecule targets a SNP in an intron of a mutated allele, and wherein the second RNA molecule targets a SNP in an intron of the mutated allele, or a sequence in an intron present in both the mutated and functional allele.

According to embodiments of the present invention, the first RNA molecule targets a sequence upstream of the promotor which is present in both a mutated and functional allele and the second RNA molecule targets a SNP or disease-causing mutation in any location of the gene.

According to embodiments of the present invention, there is provided a method comprising removing an exon containing a disease-causing mutation from a mutated allele, wherein the first RNA molecule or the first and the second RNA molecules target regions flanking an entire exon or a portion of the exon.

According to embodiments of the present invention, there is provided a method comprising removing an exon or a portion thereof from a mutant SAMD9 allele, the entire open reading frame of a mutant SAMD9 allele, or removing the entire mutant SAMD9 allele.

According to embodiments of the present invention, the first RNA molecule targets a SNP or disease-causing mutation in an exon or promoter of a mutated allele, and wherein the second RNA molecule targets a SNP in the same exon of the mutated allele, a SNP in an intron, or a sequence in an intron present in both the mutated and functional allele.

According to embodiments of the present invention, the first RNA molecule or the first and the second RNA molecules target an alternative splicing signal sequence between an exon and an intron of a mutant SAMD9 allele.

According to embodiments of the present invention, the second RNA molecule is non-discriminatory targets a sequence present in both a mutated allele and a functional allele.

The compositions and methods of the present disclosure may be utilized for treating, preventing, ameliorating, or slowing progression of an autosomal dominant genetic disorder, such as MIRAGE syndrome.

In some embodiments, a mutated allele is deactivated by delivering to a cell an RNA molecule which targets a SNP in the promoter region, the start codon, or an untranslated region (UTR) of the mutated allele.

In some embodiments, a mutated allele is inactivated by removing at least a portion of the promoter, and/or removing the start codon, and/or a portion of the UTR, and/or a polyadenylation signal. In such embodiments one RNA molecule may be designed for targeting a first SNP in the promoter or upstream to the promoter and another RNA molecule is designed to target a second SNP, which is downstream of the first SNP, and is in the promoter, in the UTR, in an intron, or in an exon. Alternatively, one RNA molecule may be designed for targeting a SNP in the promoter, upstream of the promoter, or the UTR, and another RNA molecule is designed to target a sequence which is present in an intron of both the mutated allele and the functional allele. Alternatively, one RNA molecule may be designed for targeting a sequence upstream of the promotor which is present in both the mutated and functional allele and the other guide is designed to target a SNP or disease-causing mutation in any location of the gene e.g., in an exon, intron, UTR, or downstream of the promoter.

In some embodiments, the method of deactivating a mutated allele comprises an exon skipping step comprising removing an exon containing a disease-causing mutation from the mutated allele. Removing an exon containing a disease-causing mutation in the mutated allele requires two RNA molecules which target regions flanking the entire exon or a portion of the exon. Removal of an exon containing the disease-causing mutation may be designed to eliminate the disease-causing action of the protein while allowing for expression of the remaining protein product which retains some or all of the wild-type activity. The entire open reading frame or the entire gene can be excised using two RNA molecules flanking the region desired to be excised.

In some embodiments, the method of deactivating a mutated allele comprises delivering two RNA molecules to a cell, wherein one RNA molecule targets a SNP or disease-causing mutation in an exon or promoter of the mutated allele, and wherein the other RNA molecule targets a SNP in the same of the mutated allele, a SNP in an intron, or a sequence in an intron present in both the mutated or functional allele.

Any one of, or combination of, the above-mentioned strategies for deactivating a mutant allele may be used in the context of the invention.

In embodiments of the present invention, an RNA molecule is used to direct a CRISPR nuclease to an exon or a splice site of a mutated allele in order to create a double-stranded break (DSB), leading to insertion or deletion of nucleotides by inducing an error-prone non-homologous end-joining (NHEJ) mechanism and formation of a frameshift mutation in the mutated allele. The frameshift mutation may result in, for example, inactivation or knockout of the mutated allele by generation of an early stop codon in the mutated allele and to generation of a truncated protein or to nonsense-mediated mRNA decay of the transcript of the mutant allele. In further embodiments, one RNA molecule is used to direct a CRISPR nuclease to a promotor of a mutated allele.

In some embodiments, the method of deactivating a mutated allele further comprises enhancing activity of the functional protein such as by providing a protein/peptide, a nucleic acid encoding a protein/peptide, or a small molecule such as a chemical compound, capable of activating/enhancing activity of the functional protein.

According to some embodiments, the present disclosure provides an RNA sequence (also referred to as an ‘RNA molecule’) which binds to or associates with and/or directs an RNA-guided DNA nuclease e.g., a CRISPR nuclease, to a target sequence comprising at least one nucleotide which differs between a mutated allele and a functional allele (e.g., SNP) of a gene of interest (i.e., a sequence of the mutated allele which is not present in the functional allele).

In some embodiments, the method comprises contacting a mutated allele of a gene of interest with an allele-specific RNA molecule and a CRISPR nuclease e.g., a Cas9 protein, wherein the allele-specific RNA molecule and the CRISPR nuclease associate with a nucleotide sequence of the mutated allele of the gene of interest which differs by at least one nucleotide from a nucleotide sequence of a functional allele of the gene of interest, thereby modifying or knocking-out the mutated allele.

In some embodiments, the allele-specific RNA molecule and a CRISPR nuclease is introduced to a cell encoding the gene of interest. In some embodiments, the cell encoding the gene of interest is in a mammalian subject. In some embodiments, the cell encoding the gene of interest is in a plant.

In some embodiments, the mutated allele is an allele of SAMD9 gene. In some embodiments, the RNA molecule targets a SNP which co-exists with or is genetically linked to the mutated sequence associated with MIRAGE syndrome genetic disorder. In some embodiments, the RNA molecule targets a SNP which is highly prevalent in the population and exists in the mutated allele having the mutated sequence associated with MIRAGE syndrome genetic disorder and not in the functional allele of an individual subject to be treated. In some embodiments, a disease-causing mutation within a mutated SAMD9 allele is targeted.

In some embodiments, the SNP is within an exon of the gene of interest. In such embodiments, a guide sequence portion of an RNA molecule is designed to associate with a sequence of an exon of the gene of interest.

In some embodiments, SNP is within an intron or the exon of the gene of interest. In some embodiments, the SNP is in close proximity to the splice site between an intron and an exon. In some embodiments, the close proximity to a splice site is 1, 2, 3, 4, 5, 6, 7, 8, 9, 10, 11, 12, 13, 14, 15, 16, 17, 18, 19, or 20 nucleotides upstream or downstream to the splice site. Each possibility represents a separate embodiment of the present invention. In such embodiments, a guide sequence portion of an RNA molecule may be designed to associate with a sequence of the gene of interest which comprises the splice site.

In some embodiments, the method is utilized for treating a subject having a disease phenotype resulting from the heterozygote SAMD9 gene. In such embodiments, the method results in improvement, amelioration or prevention of the disease phenotype.

Embodiments of compositions described herein include at least one CRISPR nuclease, RNA molecule(s), and a tracrRNA molecule, being effective in a subject or cells at the same time. The at least one CRISPR nuclease, RNA molecule(s), and tracrRNA may be delivered substantially at the same time or can be delivered at different times but have effect at the same time. For example, this includes delivering the CRISPR nuclease to the subject or cells before the RNA molecule and/or tracrRNA is substantially extant in the subject or cells.

In some embodiments, the cell is a stem cell. In some embodiments, the cell is an embryonic stem cell. In some embodiments, the stem cell is a hematopoietic stem/progenitor cell (HSC).

### Dominant Genetic Disorders

One of skill in the art will appreciate that all subjects with any type of heterozygote genetic disorder (e.g., dominant genetic disorder) may be subjected to the methods described herein. In one embodiment, the present invention may be used to target a gene involved in, associated with, or causative of dominant genetic disorders such as, for example, MIRAGE syndrome. In some embodiments, the dominant genetic disorder is MIRAGE syndrome. In some embodiments, the target gene is the SAMD9 gene (Entrez Gene, gene ID No: 54809). Non-limiting examples of mutations previously characterized as gain of function mutations associated with MIRAGE syndrome phenotype include: 7:93101391_C_G, 7:93101435_G_A, 7:93101512_C_T, 7:93101540_C_A, 7:93101615_T_C, 7:93101761_AGTT_A, 7:93101900_G_A, 7:93102220_C_T, 7:93102221_G_A, 7:93102242_G_T, 7:93102259_G_A, 7:93102368_T_C, 7:93102514_G_A, 7:93102593_G_A, 7:93102692_C_G, 7:93102717_G_T, 7:93103150_AT_GA, 7:93103151_T_C, 7:93103153_C_T, 7:93103178_C_T, 7:93103253_C_A, 7:93103393_C_T, 7:93103588_C_CT, 7:93103598_T_A, 7:93103628_G_A, 7:93103679_C_T, 7:93103765_G_A, 7:93103772_G_T, 7:93103793_C_T, 7:93103851_G_T, 7:93103900_A_G, 7:93103916_T_C, 7:93103933_G_T, 7:93103938_GT_G, 7:93104044_C_T, 7:93104176_A_G, 7:93104296_ATT_A, 7:93104449_T_A, 7:93104694_TAC_GCA, 7:93104695_AC_GA, 7:93104722_C_T, 7:93105043_A_G, 7:93105103_A_G, 7:93105122_G_A, 7:93105295_A_G, 7:93105436_C_T, 7:93105485_T_G, 7:93105638_G_A, 7:93105781_C_T, 7:93105965_CT_C, 7:93106002_GT_G, 7:93106003_T_A, and 7:93106096_A_G.

SAMD9 editing strategies include, but are not limited to, (1) truncation; (2) inhibiting expression of a mutated SAMD9 allele; (3) excision of the mutated coding exon or polyadenylation signal; and (4) targeting a SAMD9 mutation to induce a frameshift; and (5) inducing homology directed repair (HDR) to introduce a synthetic splice site upstream of the coding exon with a donor molecule.

Truncation can be achieved by several approaches. For example, excision may be achieved by targeting the mutant SAMD9 allele with two different RNA molecules, e.g. single guide RNA molecules or “sgRNAs”. At least one RNA molecule is preferably allele-specific. Alternatively, truncation may also be achieved by targeting a SNP within the coding exon of a mutant SAMD9 allele using a single guide RNA molecule.

In another approach, truncation can be achieved by introducing a splice acceptor by HDR. A splice acceptor sequence can be introduced using, for example, a double-stranded donor oligonucleotide (dsODN) template that will be introduced in Intron 2 before the coding exon.

In another editing strategy, expression of a mutated SAMD9 allele may be inhibited. This can be achieved by excising the polyadenylation signal in the 3′UTR region, which leads to an unstable transcript.

According to embodiments of the present invention, there is provided an RNA molecule comprising a guide sequence portion (e.g., a targeting sequence) comprising a nucleotide sequence that is fully or partially complementary to a target sequence in a SNP position (REF/SNP sequence) located in or near a mutated allele of the SAMD9 gene. In some embodiments, the guide sequence portion of the RNA molecule consists of 16, 17, 18, 19, 20, 21, 22, 23, 24, 25, 26, or more than 26 nucleotides. In such embodiments, the guide sequence portion comprises a sequence that is the same as or differs by no more than 1, 2, or 3 nucleotides from a sequence set forth in Table 1. Each possibility represents a separate embodiment. In some embodiments the guide sequence portion is configured to target a CRISPR nuclease to a target sequence and provide a cleavage event, by a CRISPR nuclease complexed therewith, selected from a double-strand break and a single-strand break within 500, 400, 300, 200, 100, 50, 25, or 10 nucleotides of a SAMD9 target site. In some embodiments, the cleavage event enables non-sense mediated decay of the SAMD9 gene. In some embodiments, the RNA molecule is a guide RNA molecule such as a crRNA molecule or a single guide RNA molecule.

In some embodiments, the target sequence of a mutated allele of SAMD9 gene is altered (e.g., by introduction of an NHEJ-mediated indel (e.g., insertion or deletion), and results in reduction or elimination of expression of the gene product encoded by the mutant allele of SAMD9 gene. In some embodiments, the reduction or elimination of expression is due to non-sense mediated mRNA decay such as due to immature stop codon. In some embodiments, the reduction or elimination of expression is due to expression of a truncated form of the SAMD9 gene product. In some embodiments, the guide sequence portion is complementary to a target sequence in a SNP position located in the coding exon (Exon 3) or from 7, 30, or 50 base pairs upstream to 7, 30, or 50 base pairs downstream of the coding exon (Exon 3) of the mutated allele of the SAMD9 gene. Each possibility represents a separate embodiment. In such embodiments, the guide sequence portion comprises a sequence that targets a SNP position selected from: rs10279499, rs78564070, rs6965201, or rs6969691. In such embodiments, the guide sequence portion comprises a sequence that is the same as or differs by no more than 1, 2, or 3 nucleotides from a sequence selected from any of SEQ ID NOs: 9909-10964, 5744, 5746, 5765, 5772, 5776, 5780, 5786, 5789, 5791, 5796, 5798, 5815, 5818, 5825, 5840, 5843, 5845, 5852, 5854, 5869, 5872, 5880, 5891, 5899, 6678-6679, 6681, 6688, 6692, 6696, 6710, 6714, or 6722, or from a sequence set forth in any of the SEQ ID NOs listed as targeting rs10279499, rs78564070, rs6965201, or rs6969691 in Table 1.

In some embodiments, the guide sequence portion is complementary to target a pathogenic mutation located in the coding exon (Exon 3) of the mutated allele of the SAMD9 gene to reduce or eliminate expression of the mutated SAMD9 allele. In such embodiments, the guide sequence portion comprises a sequence that targets a mutation selected from: 7:93101391_C_G, 7:93101435_G_A, 7:93101512_C_T, 7:93101540_C_A, 7:93101615_T_C, 7:93101761_AGTT_A, 7:93101900_G_A, 7:93102220_C_T, 7:93102221_G_A, 7:93102242_G_T, 7:93102259_G_A, 7:93102368_T_C, 7:93102514_G_A, 7:93102593_G_A, 7:93102692_C_G, 7:93102717_G_T, 7:93103150_AT_GA, 7:93103151_T_C, 7:93103153_C_T, 7:93103178_C_T, 7:93103253_C_A, 7:93103393_C_T, 7:93103588_C_CT, 7:93103598_T_A, 7:93103628_G_A, 7:93103679_C_T, 7:93103765_G_A, 7:93103772_G_T, 7:93103793_C_T, 7:93103851_G_T, 7:93103900_A_G, 7:93103916_T_C, 7:93103933_G_T, 7:93103938_GT_G, 7:93104044_C_T, 7:93104176_A _G, 7:93104296_ATT_A, 7:93104449_T_A, 7:93104694_TAC_GCA, 7:93104695_AC_GA, 7:93104722_C_T, 7:93105043_A _G, 7:93105103_A_G, 7:93105122_G_A, 7:93105295_A_G, 7:93105436_C_T, 7:93105485_T_G, 7:93105638_G_A, 7:93105781_C_T, 7:93105965_CT_C, 7:93106002_GT_G, 7:93106003_T_A, and 7:93106096_A_G. In some embodiments, the reduction or elimination of expression is due to non-sense mediated mRNA decay such as due to immature stop codon. In some embodiments, the reduction or elimination of expression is due to expression of a truncated form of the SAMD9 gene product of the mutant allele. In some embodiments, a donor HDR is further introduced to correct the sequence of the mutant allele of the SAMD9 gene.

In some embodiments, the expression of the coding exon of a mutated allele of SAMD9 gene is eliminated by introducing a splice acceptor directly upstream of the coding exon (e.g., to intron 2) of the mutated allele. In some embodiments, the target sequence is a SNP position in intron 2 of a mutated allele of SAMD9 gene, to which a splice acceptor is introduced such as by HDR following DSB by a RNA targeted DNA Nuclease, this results in reduction or elimination of expression of the gene product encoded by the mutant allele of SAMD9 gene. In such embodiments, the SNP position in intron 2 is any one of: rs2374627, rs12668221, rs28673511, rs10229795, rs7785869, rs67885698, rs6957832, or rs12704664. In such embodiments, the first guide sequence portion comprises a sequence that is the same as or differs by no more than 1, 2, or 3 nucleotides from a sequence set forth in any of the SEQ ID NOs listed as targeting rs2374627, rs12668221, rs28673511, rs10229795, rs7785869, rs67885698, rs6957832, or rs12704664 in Table 1. A person skilled in the art will appreciate that many splice acceptor sequences can be utilized.

In some embodiments, the coding exon of a mutated allele of SAMD9 gene is completely or partially excised to reduce or eliminate expression of the mutant allele of the SAMD9 gene. In such embodiments at least two different guide sequence portions are utilized, a first guide sequence portion is a discriminatory guide targeting only a mutated allele of the SAMD9 gene, and a second guide sequence portion is non-discriminatory and targets non-coding regions of both alleles of the SAMD9 gene.

In some embodiments, the first guide sequence portion is complementary to a target sequence in a SNP position located in intron 2 of the mutated allele of the SAMD9 gene, and the second guide sequence portion is complementary to a target sequence in the 3′ UTR or in the intergenic region of the SAMD9 gene. In such embodiments, the SNP position is any one of rs2374627, rs12668221, rs28673511, rs10229795, rs7785869, rs67885698, rs6957832, or rs12704664. In such embodiments, the first guide sequence portion comprises a sequence that is the same as or differs by no more than 1, 2, or 3 nucleotides from a sequence set forth in any of the SEQ ID NOs listed as targeting rs2374627, rs12668221, rs28673511, rs10229795, rs7785869, rs67885698, rs6957832, or rs12704664 in Table 1.

In some embodiments, the first guide sequence portion is complementary to a target sequence in a SNP position located in intergenic region of the mutated allele of the SAMD9 gene, and the second guide sequence portion is complementary to a target sequence in intron 2 of the SAMD9 gene. In such embodiments, the SNP position is any one of: rs71562815, rs12333976, rs3839837, rs201891812, rs73216083, rs542330750, rs7798225, rs10953089, rs10953090, or rs10277338. In such embodiments, the first guide sequence portion comprises a sequence that is the same as or differs by no more than 1, 2, or 3 nucleotides from a sequence set forth in any of the SEQ ID NOs listed as targeting rs71562815, rs12333976, rs3839837, rs201891812, rs73216083, rs542330750, rs7798225, rs10953089, rs10953090, or rs10277338 in Table 1. In such embodiments, the second guide sequence portion comprises a sequence that is the same as or differs by no more than 1, 2, or 3 nucleotides from a sequence set forth in any of SEQ ID Nos: 18323-20640, or from the SEQ ID NOs listed as targeting intron 2 of the SAMD9 gene in Table 1.

In some embodiments, the first guide sequence portion is complementary to a target sequence in a SNP position located in 3′ UTR of the mutated allele of the SAMD9 gene, and the second guide sequence portion is complementary to a target sequence in intron 2 of the SAMD9 gene. In such embodiments, the SNP position in the 3′UTR is any one of rs71562815, rs12333976, or rs3839837. In such embodiments, the first guide sequence portion comprises a sequence that is the same as or differs by no more than 1, 2, or 3 nucleotides from a sequence set forth in any of SEQ ID Nos: 9471-9908, or from the SEQ ID NOs listed as targeting rs71562815, rs12333976, or rs3839837 in Table 1. In such embodiments, the second guide sequence portion comprises a sequence that is the same as or differs by no more than 1, 2, or 3 nucleotides from a sequence set forth in any of SEQ ID Nos: 18323-20640.

In some embodiments, the polyadenylation signal (PolyAS) of SAMD9 gene of the mutated allele of the SAMD9 gene is completely or partially excised to destabilize the mutated allele to reduce or eliminate expression of the mutant allele.

In some embodiments, the first guide sequence portion is complementary to a target sequence in a SNP position located in 3′ UTR of the mutated allele of the SAMD9 gene, and the second guide sequence portion is complementary to a target sequence in the intergenic region of the SAMD9 gene. In such embodiments, the SNP position in the 3′UTR is any one of rs71562815, rs12333976, or rs3839837. In such embodiments, the first guide sequence portion comprises a sequence that is the same as or differs by no more than 1, 2, or 3 nucleotides from a sequence set forth in any of SEQ ID Nos: 9471-9908, or from the SEQ ID NOs listed as targeting rs71562815, rs12333976, or rs3839837 in Table 1.

In some embodiments, the first guide sequence portion is complementary to a target sequence in a SNP position located in intergenic region of the mutated allele of the SAMD9 gene, and the second guide sequence portion is complementary to a target sequence in the 3′ UTR of the SAMD9 gene. In such embodiments, the SNP position in the intergenic region is any one of rs71562815, rs12333976, rs3839837, rs201891812, rs73216083, rs542330750, rs7798225, rs10953089, rs10953090, or rs10277338. In such embodiments, the first guide sequence portion comprises a sequence that is the same as or differs by no more than 1, 2, or 3 nucleotides from a sequence set forth in any of the SEQ ID NOs listed as targeting rs71562815, rs12333976, rs3839837, rs201891812, rs73216083, rs542330750, rs7798225, rs10953089, rs10953090, or rs10277338 in Table 1. In such embodiments, the second guide sequence portion comprises a sequence that is the same as or differs by no more than 1, 2, or 3 nucleotides from a sequence set forth in any of SEQ ID Nos: 12756-17909, or from the SEQ ID NOs listed as targeting the 3′ UTR of the SAMD9 gene in Table 1.

### CRISPR Nucleases and PAM Recognition

In some embodiments, the sequence specific nuclease is selected from CRISPR nucleases, or a functional variant thereof. In some embodiments, the sequence specific nuclease is an RNA guided DNA nuclease. In such embodiments, the RNA sequence which guides the RNA guided DNA nuclease (e.g., Cpfl) binds to and/or directs the RNA guided DNA nuclease to the sequence comprising at least one nucleotide which differs between a mutated allele and its counterpart functional allele (e.g., SNP). In some embodiments, the CRISPR complex does not further comprise a tracrRNA. In a non-limiting example, in which the RNA guided DNA nuclease is a CRISPR protein, the at least one nucleotide which differs between the dominant mutated allele and the functional allele may be within the PAM site and/or proximal to the PAM site within the region that the RNA molecule is designed to hybridize to. A skilled artisan will appreciate that RNA molecules can be engineered to bind to a target of choice in a genome by commonly known methods in the art.

The term “PAM” as used herein refers to a nucleotide sequence of a target DNA located in proximity to the targeted DNA sequence and recognized by the CRISPR nuclease complex. The PAM sequence may differ depending on the nuclease identity. In addition, there are CRISPR nucleases that can target almost all PAMs. In some embodiments of the present invention, a CRISPR system utilizes one or more RNA molecules having a guide sequence portion to direct a CRISPR nuclease to a target DNA site via Watson-Crick base-pairing between the guide sequence portion and the protospacer on the target DNA site, which is next to the protospacer adjacent motif (PAM), which is an additional requirement for target recognition. The CRISPR nuclease then mediates cleavage of the target DNA site to create a double-stranded break within the protospacer. In a non-limiting example, a type II CRISPR system utilizes a mature crRNA:tracrRNA complex that directs the CRISPR nuclease, e.g. Cas9 to the target DNA the target DNA via Watson-Crick base-pairing between the guide sequence portion of the crRNA and the protospacer on the target DNA next to the protospacer adjacent motif (PAM). A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NRRH for SpCas9-NRRH variant, wherein N is any nucleobase, R is A or G and H is A, C, or T; NRTH for SpCas9-NRTH variant, wherein N is any nucleobase, R is A or G and H is A, C, or T; NRCH for SpCas9-NRCH variant, wherein N is any nucleobase, R is A or G and H is A, C, or T; NG for SpG variant of SpCas9 wherein N is any nucleobase; NG or NA for SpCas9-NG variant of SpCas9 wherein N is any nucleobase; NR or NRN or NYN for SpRY variant of SpCas9, wherein N is any nucleobase, R is A or G and Y is C or T; NNG for Streptococcus canis Cas9 variant (ScCas9), wherein N is any nucleobase; NNNRRT for SaKKH-Cas9 variant of Staphylococcus aureus (SaCas9), wherein N is any nucleobase, and R is A or G; NNNNGATT for Neisseria meningitidis (NmCas9), wherein N is any nucleobase; TTN for Alicyclobacillus acidiphilus Cas12b (AacCas12b), wherein N is any nucleobase; or TTTV for Cpfl, wherein V is A, C or G. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Pat. Publication No. 2015/0211023, incorporated herein by reference.

CRISPR systems that may be used in the practice of the invention vary greatly. CRISPR systems can be a type I, a type II, or a type III system. Non- limiting examples of suitable CRISPR proteins include Cas3, Cas4, Cas5, Cas5e (or CasD), Cas6, Cas6e, Cas6f, Cas7, Cas8al, Cas8a2, Cas8b, Cas8c, Cas9, Cas10, Cas1 Od, CasF, CasG, CasH, Csy1, Csy2, Csy3, Csel (or CasA), Cse2 (or CasB), Cse3 (or CasE), Cse4 (or CasC), Cscl, Csc2, Csa5, Csn2, Csm2, Csm3, Csm4, Csm5, Csm6, Cmrl, Cmr3, Cmr4, Cmr5, Cmr6, Csbl, Csb2, Csb3,Csxl7, Csxl4, Csxl0, Csxl6, CsaX, Csx3, Cszl, Csxl5, Csf1, Csf2, Csf3, Csf4, and Cul966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant, SpCas9 EQR variant, or SpCas9 VRER variant may also be used in the context of the invention.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpfl and its homologs and orthologs, may be used in the compositions of the present invention. Additional CRISPR nucleases may also be used, for example, the nucleases described in PCT International Application Publication Nos. WO2020/223514 and WO2020/223553, which are hereby incorporated by reference.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpfl. Cpfl is a single RNA-guided endonuclease which utilizes a T-rich protospacer-adjacent motif. Cpfl cleaves DNA via a staggered DNA double-stranded break. Two Cpfl enzymes from Acidaminococcus and Lachnospiraceae have been shown to carry out efficient genome-editing activity in human cells. (See Zetsche et al., 2015).

Thus, an RNA guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpfl and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine, 2′-O-methylcytidine, 5-carboxymethylaminomethyl-2-thiouridine, 5-carboxymethylammomethyluridine, dihydrouridine, 2′-O-methylpseudouridine, “beta, D-galactosylqueuosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethylundine, 5-methoxyaminomethyl-2-thiouridine, “beta, D-mannosylqueuosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofmanosylpurine-6-yl)-carbamoyl)threonine, 2′-O-methyl-5-methyluridine, 2′-O-methyluridine, wybutosine, “3-(3-amino-3-carboxy-propyl)uridine, (acp3)u”, 2′-0-methyl (M), 3′-phosphorothioate (MS), 3′-thioPACE (MSP), pseudouridine, or 1-methyl pseudo-uridine. Each possibility represents a separate embodiment of the present invention.

### Guide Sequences Which Specifically Target a Mutant Allele

A given gene may contain thousands of SNPs. Utilizing a 25 base pair target window for targeting each SNP in a gene would require hundreds of thousands of guide sequences. Any given guide sequence when utilized to target a SNP may result in degradation of the guide sequence, limited activity, no activity, or off-target effects. Accordingly, suitable guide sequences are necessary for targeting a given gene. By the present invention, a novel set of guide sequences have been identified for knocking out expression of a mutated SAMD9 protein, inactivating a mutant SAMD9 gene allele, and treating MIRAGE syndrome.

The present disclosure provides guide sequences capable of specifically targeting a mutated allele for inactivation while leaving the functional allele unmodified. The guide sequences of the present invention are designed to, and are most likely to, specifically differentiate between a mutated allele and a functional allele. Of all possible guide sequences which target a mutated allele desired to be inactivated, the specific guide sequences disclosed herein are specifically effective to function with the disclosed embodiments.

Briefly, the guide sequences may have properties as follows: (1) target SNP/insertion/deletion/indel with a high prevalence in the general population, in a specific ethnic population or in a patient population is above 1% and the SNP/insertion/deletion/indel heterozygosity rate in the same population is above 1%; (2) target a location of a SNP/insertion/deletion/indel proximal to a portion of the gene e.g., within 5k bases of any portion of the gene, for example, a promoter, a UTR, an exon or an intron; and (3) target a mutant allele using an RNA molecule which targets a founder or common pathogenic mutations for the disease/gene. In some embodiments, the prevalence of the SNP/insertion/deletion/indel in the general population, in a specific ethnic population or in a patient population is above 1%, 2%, 3%, 4%, 5%, 6%, 7%, 8%, 9%, 10%, 11%, 12%, 13%, 14%, or 15% and the SNP/insertion/deletion/indel heterozygosity rate in the same population is above 1%, 2%, 3%, 4%, 5%, 6%, 7%, 8%, 9%, 10%, 11%, 12%, 13%, 14%, or 15%. Each possibility represents a separate embodiment and may be combined at will.

For each gene, according to SNP/insertion/deletion/indel any one of the following strategies may be used to deactivate the mutated allele: (1) Knockout strategy using one RNA molecule - one RNA molecule is utilized to direct a CRISPR nuclease to a mutated allele and create a double-strand break (DSB) leading to formation of a frameshift mutation in an exon or in a splice site region of the mutated allele, or alternatively one RNA molecule is utilized to target an intron of a mutated allele leading to introduction of a synthetic splice donor by HDR; (2) Excision of the coding exon using two RNA molecules, for example, a first RNA molecule targets a SNP position of an exon of the mutated allele and a second, non-discriminatory RNA molecule targets a sequence within flanking the exon; (3) Excision of the polyadenylation signal using two RNA molecules, for example, a first RNA molecule targets a SNP position in the 3′UTR and a second, non-discriminatory RNA molecule targets a sequences downstream of the polyadenylation signal. Alternatively, the first RNA molecule targets a SNP position located downstream of the polyadenylation signal and the second, non-discriminatory RNA molecule targets a sequence upstream of the polyadenylation signal.

Based on the locations of identified SNPs/insertions/deletions/indels for each mutant allele, any one of, or a combination of, the above-mentioned methods to deactivate the mutant allele may be utilized.

In some embodiments of the present invention, one RNA molecule is used to target a SNP, and the location of the SNP is in an exon or in close proximity (e.g., within 20 base pairs) to a splice site between an intron and an exon. In some embodiments, two RNA molecules are used and may target two SNPs, such that the first SNP is upstream of the first exon e.g., within the 5′ untranslated region, within the promoter, or within the first two kilobases 5′ of the transcription start site, and the second SNP is downstream of the first SNP e.g., within the first two kilobases 5′ of the transcription start site, within an intron, or within an exon.

Guide sequences of the present invention may target a SNP in the upstream portion of the targeted gene, preferably upstream of the last exon of the targeted gene. Guide sequences may target a SNP upstream to the first exon, for example within the 5′ untranslated region, within the promoter, or within the first 4-5 kilobases 5′ of the transcription start site.

Guide sequences of the present invention may also target a SNP within close proximity (e.g., within 50 base pairs, more preferably with 20 base pairs) to a known protospacer adjacent motif (PAM) site.

Guide sequences of the present invention may: (1) target a heterozygous SNP for the targeted gene; (2) target a heterozygous SNP upstream or downstream of the gene; (3) target a SNP with a prevalence of the SNP/insertion/deletion/indel in the general population, in a specific ethnic population, or in a patient population above 1%; (4) have a guanine-cytosine content of greater than 30% and less than 85%; (5) have no repeat of four or more thymine/uracil or eight or more guanine, cytosine, or adenine; and (6) have low or no off-targeting identified by off-target analysis. Guide sequences of the present invention may satisfy any one of the above criteria and are most likely to differentiate between a mutated allele from its corresponding functional allele.

In some embodiments of the present invention, a SNP targeted by an RNA molecule may be upstream or downstream of the gene. In some embodiments of the present invention, the SNP is within 4,000 base pairs of the gene.

in some embodiments of the present invention, at least one nucleotide which differs between the mutated allele and the functional allele is upstream, downstream or within the sequence of the disease-causing mutation of the gene of interest. The at least one nucleotide which differs between the mutated allele and the functional allele may be within an exon or within an intron of the gene of interest. In some embodiments, the at least one nucleotide which differs between the mutated allele and the functional allele is within an exon of the gene of interest. In some embodiments, the at least one nucleotide which differs between the mutated allele and the functional allele is within an intron or the exon of the gene of interest, in close proximity to the splice site between the intron and the exon e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotides upstream or downstream to the splice site. Each possibility represents a separate embodiment.

In some embodiments, the at least one nucleotide is a single nucleotide polymorphism (SNP). In some embodiments, each of the nucleotide variants of the SNP may be expressed in the mutated allele. In some embodiments, the SNP may be a founder or common pathogenic mutation.

Guide sequences may target a SNP which has both (1) a high prevalence in the general population e.g., above 1% in the population; and (2) a high heterozygosity rate in the population, e.g., above 1%. Guide sequences may target a SNP that is globally distributed. A SNP may be a founder or common pathogenic mutation. In some embodiments, the prevalence in the general population is above 1%, 2%, 3%, 4%, 5%, 6%, 7%, 8%, 9%, 10%, 11%, 12%, 13%, 14%, or 15%. Each possibility represents a separate embodiment. In some embodiments, the heterozygosity rate in the population is above 1%, 2%, 3%, 4%, 5%, 6%, 7%, 8%, 9%, 10%, 11%, 12%, 13%, 14%, or 15%. Each possibility represents a separate embodiment.

In some embodiments, the at least one nucleotide which differs between the mutated allele and the functional allele is linked to/co-exists with the disease-causing mutation in high prevalence in a population. In such embodiments, “high prevalence” refers to at least 92%, 93%, 94%, 95%, 96%, 97%, 98%, 99%, or 100%. Each possibility represents a separate embodiment of the present invention. In one embodiment, the at least one nucleotide which differs between the mutated allele and the functional allele, is a disease-associated mutation. In some embodiments, the SNP is highly prevalent in the population. In such embodiments, “highly prevalent” refers to at least 10%, 11%, 12%, 13%, 14%, 15%, 20%, 30%, 40%, 50%, 60%, or 70% of a population. Each possibility represents a separate embodiment of the present invention.

### Delivery to Cells

The RNA molecule compositions described herein may be delivered to a target cell by any suitable means. RNA molecule compositions of the present invention may be targeted to any cell which contains and/or expresses a mutated allele, including any mammalian or plant cell. For example, in one embodiment the RNA molecule specifically targets a mutated SAMD9 allele and the target cell is an HSC. The delivery to the cell may be performed in-vitro, ex-vivo, or in-vivo. Further, the nucleic acid compositions described herein may be delivered as one or more of DNA molecules, RNA molecules, Ribonucleoproteins (RNP), nucleic acid vectors, or any combination thereof.

In some embodiments, the RNA molecule comprises a chemical modification. Non-limiting examples of suitable chemical modifications include 2′-0-methyl (M), 2′-0-methyl, 3′phosphorothioate (MS) or 2′-0-methyl, 3 ‘thioPACE (MSP), pseudouridine, and 1-methyl pseudouridine. Each possibility represents a separate embodiment of the present invention.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992); Nabel & Felgner (1993); Mitani & Caskey (1993); Dillon (1993); Miller (1992); Van Brunt (1988); Vigne (1995); Kremer & Perricaudet (1995); Haddada et al. (1995); and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo, ex vivo, or in vitro delivery method. (See Zuris et al. (2015); see also Coelho et al. (2013); Judge et al. (2006); and Basha et al. (2011)).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa.RTM. Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. No. 5,049,386, U.S. Pat. No. 4,946,787; and U.S. Pat. No. 4,897,355, and lipofection reagents are sold commercially (e.g., Transfectam.TM., Lipofectin.TM. and Lipofectamine.TM. RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., (1995); Behr et al., (1994); Remy et al. (1994); Gao and Huang (1995); Ahmad and Allen (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992); Johann et al. (1992); Sommerfelt et al. (1990); Wilson et al. (1989); Miller et al. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997); Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravitreal, intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow, and spleen) from a patient for culture, nucleic acid transfer to the cultured cells (e.g., hematopoietic stem cells), followed by grafting the cells to a target tissue (e.g., bone marrow, and spleen) of the patient. In some embodiments, the stem cell or hematopoietic stem cell may be further treated with a viability enhancer.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Suitable cells include, but are not limited to, eukaryotic cells and/or cell lines. Non-limiting examples of such cells or cell lines generated from such cells include COS, CHO (e.g., CHO--S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), perC6 cells, any plant cell (differentiated or undifferentiated), as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with a guided nuclease system (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma, and TNF-alpha are known (as a non-limiting example see, Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+(panB cells), GR-1 (granulocytes), and Iad (differentiated antigen presenting cells) (as a non-limiting example, see Inaba et al., 1992). Stem cells that have been modified may also be used in some embodiments.

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Pat. Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington’s Pharmaceutical Sciences, 17th ed., 1989).

In accordance with some embodiments, there is provided an RNA molecule which binds to/ associates with and/or directs the RNA guided DNA nuclease to a sequence comprising at least one nucleotide which differs between a mutated allele and a functional allele (e.g., SNP) of a gene of interest (i.e., a sequence of the mutated allele which is not present in the functional allele). The sequence may be within the disease associated mutation. The sequence may be upstream or downstream to the disease associated mutation. Any sequence difference between the mutated allele and the functional allele may be targeted by an RNA molecule of the present invention to inactivate the mutant allele, or otherwise disable its dominant disease-causing effects, while preserving the activity of the functional allele.

The disclosed compositions and methods may also be used in the manufacture of a medicament for treating dominant genetic disorders in a patient.

### Mechanisms of Action for SAMD9 Knockout Methods

Without being bound by any theory or mechanism, the instant invention may be utilized to apply a CRISPR nuclease to process a mutated pathogenic SAMD9 allele and not a functional SAMD9 allele, such as to prevent expression of the mutated pathogenic allele or to produce a truncated non-pathogenic peptide from the mutated pathogenic allele, in order to prevent MIRAGE syndrome. A specific guide sequence may be selected from Table 1 based on the targeted SNP and the type of CRISPR nuclease used (required PAM sequence).

The SAMD9 gene is located in chromosome 7 and encodes the SAMD9 protein. One optional strategy to knockout a mutated SAMD9 allele is to truncate it by targeting a SNP position in a coding exon of SAMD9 using one RNA molecule. Truncation may also be achieved by excision of an exon using two RNA molecules, wherein one of the RNA molecules targets an allele specific sequence in a SNP position and the other RNA molecule is a non-discriminatory guide that targets a sequence flanking the coding exon. Another optional strategy to knockout SAMD9 is by excision of its polyadenylation signal using a first RNA molecule to target a SNP position in the 3′UTR and a second, non-discriminatory RNA to target downstream of the polyadenylation signal. Alternatively, excision of the SAMD9 polyadenylation signal may be performed by using a first RNA molecule to target a SNP position located downstream of the polyadenylation signal and a second, non-discriminatory RNA molecule to target a sequence upstream of the polyadenylation signal. Excision of a portion of a mutated SAMD9 allele may also be achieved by mediating a DSB in an intron flanking the desired exon and a region downstream of the desired exon. Yet another optional strategy is to introduce by HDR a synthetic splice acceptor site in Intron 2, such that the coding exon is no longer expressed.

### Examples of RNA Guide Sequences Which Specifically Target Mutated Alleles of SAMD9 Gene

Disclosures which include sequences that may interact with a SARM1 sequence in some form include PCT International Application Publication Nos. WO2020/191171, WO2013/081864, WO2017/072590, WO2019/081982, WO2018/154412, Japanese Application Publication No. 2008/504843, and U.S. Publication Nos. 2006/0019284 and 2006/0003322, each of which are hereby incorporated by reference. Although a large number of guide sequences can be designed to target a mutated allele, the nucleotide sequences described in Tables 2 identified by SEQ ID NOs: 1-20640 below were specifically selected to effectively implement the methods set forth herein and to effectively discriminate between alleles.

Table 1 shows guide sequences designed for use as described in the embodiments above to associate with different SNPs or pathogenic mutations within a sequence of a mutated SAMD9 allele. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), SpRY (PAM SEQ: NRN or NYN), NmCas9WT (PAM SEQ: NNNNGATT), Cpfl (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

TABLE 1

Guide sequence portions designed to associate with specific SAMD9 gene targets

| Target | SEQ ID NOs: of 20-nucleotide guide sequence portions | SEQ ID NOs: of 21-nucleotide guide sequence portions | SEQ ID NOs: of 22-nucleotide guide sequence portions |
|---|---|---|---|
| 7:93101391_C_G | 1-52 | 53-106 | 107-162 |
| 7:93101435_G_A | 163-214 | 215-268 | 269-324 |
| 7:93101512_C_T | 325-376 | 377-430 | 431-486 |
| 7:93101540_C_A | 487-538 | 539-592 | 593-648 |
| 7:93101615_T_C | 649-700 | 701-754 | 755-810 |
| 7:93101761_AGTT_A | 811-852 | 853-896 | 897-942 |
| 7:93101900_G_A | 943-994 | 995-1048 | 1049-1104 |
| 7:93102220_C_T | 1105-1156 | 1157-1210 | 1211-1266 |
| 7:93102221_G_A | 1106-1107, 1109, 1113-1114, 1120, 1127, 1142, 1146, 1154, 1267-1308 | 1158, 1161, 1165, 1172, 1179, 1190, 1194, 1198-1199, 1208, 1309-1352 | 1212, 1219, 1226, 1244, 1248, 1252-1253, 1259, 1262-1263, 1353-1398 |
| 7:93102242_G_T | 1399-1444 | 1445-1486 | 1487-1536 |
| 7:93102259_G_A | 1537-1588 | 1589-1641 | 1642-1697 |
| 7:93102368_T_C | 1698-1749 | 1750-1803 | 1804-1859 |
| 7:93102514_G_A | 1860-1911 | 1912-1963 | 1964-2019 |
| 7:93102593_G_A | 2020-2071 | 2072-2125 | 2126-2181 |
| 7:93102692_C_G | 2182-2233 | 2234-2287 | 2288-2343 |
| 7:93102717_G_T | 2344-2385 | 2386-2421 | 2422-2463 |
| 7:93103150_AT_GA | 2464-2515 | 2516-2569 | 2570-2625 |
| 7:93103151_T_C | 2466, 2472, 2485-2486, 2496, 2499, 2503, 2508, 2513, 2515, 2626-2667 | 2518, 2524, 2532, 2539, 2543, 2553, 2557, 2562, 2567, 2569, 2668-2711 | 2572, 2576, 2579, 2581, 2587, 2594, 2598, 2613, 2623, 2625, 2712-2757 |
| 7:93103153_C_T | 2485-2486, 2503, 2508, 2513, 2515, 2640, 2665, 2758-2801 | 2524, 2532, 2539, 2557, 2562, 2569, 2673, 2689, 2802-2847 | 2572, 2576, 2579, 2587, 2594, 2625, 2736, 2752, 2848-2895 |
| 7:93103178_C_T | 2896-2947 | 2948-3001 | 3002-3057 |
| 7:93103253_C_A | 3058-3109 | 3110-3163 | 3164-3219 |
| 7:93103393_C_T | 3220-3271 | 3272-3324 | 3325-3379 |
| 7:93103588_C_CT | 3380-3427 | 3428-3477 | 3478-3529 |
| 7:93103598_T_A | 3530-3581 | 3582-3635 | 3636-3691 |
| 7:93103628_G_A | 3692-3743 | 3744-3787 | 3788-3835 |
| 7:93103679_C_T | 3836-3887 | 3888-3937 | 3938-3991 |
| 7:93103765_G_A | 3992-4043 | 4044-4097 | 4098-4153 |
| 7:93103772_G_T | 4154-4205 | 4206-4259 | 4260-4315 |
| 7:93103793_C_T | 4316-4367 | 4368-4421 | 4422-4477 |
| 7:93103851_G_T | 4478-4529 | 4530-4583 | 4584-4639 |
| 7:93103900_A_G | 4640-4691 | 4692-4745 | 4746-4801 |
| 7:93103916_T_C | 4802-4853 | 4854-4907 | 4908-4963 |
| 7:93103933_G_T | 4964-5015 | 5016-5069 | 5070-5125 |
| 7:93103938_GT_G | 5126-5169 | 5170-5215 | 5216-5263 |
| 7:93104044_C_T | 5264-5315 | 5316-5369 | 5370-5425 |
| 7:93104176_A_G | 5426-5477 | 5478-5531 | 5532-5587 |
| 7:93104296_ATT_A | 5588-5635 | 5636-5685 | 5686-5737 |
| 7:93104449_T_A | 5738-5789 | 5790-5843 | 5844-5899 |
| 7:93104694_TAC_GCA 7:93104695_AC_GA | 5900-5951 5900, 5906, 5910, 5921, 5924, 5926-5927, 5932, 5939, 5941, 6062-6103 | 5952-6005 5958, 5962, 5973, 5976, 5978, 5980, 5985, 5992, 5994, 6001, 6104-6147 | 6006-6061 6012, 6014, 6029, 6032, 6034, 6036, 6041, 6048, 6057, 6060, 6148-6193 |
| 7:93104722_C_T | 6194-6245 | 6246-6299 | 6300-6355 |
| 7:93105043_A_G | 6356-6407 | 6408-6459 | 6460-6515 |
| 7:93105103_A_G | 6516-6567 | 6568-6618 | 6619-6673 |
| 7:93105122_G_A | 6674-6707 | 6708-6712 | 6713-6723 |
| 7:93105295_A_G | 6724-6775 | 6776-6829 | 6830-6885 |
| 7:93105436_C_T | 6886-6937 | 6938-6991 | 6992-7047 |
| 7:93105485_T_G | 7048-7099 | 7100-7153 | 7154-7209 |
| 7:93105638_G_A | 7210-7261 | 7262-7311 | 7312-7364 |
| 7:93105781_C_T | 7365-7416 | 7417-7470 | 7471-7526 |
| 7:93105965_CT_C | 7527-7571 | 7572-7617 | 7618-7663 |
| 7:93106002_GT_G | 7664-7713 | 7714-7765 | 7766-7819 |
| 7:93106003_T_A | 7668, 7672, 7678, 7691-7692, 7697-7699, 7703, 7705, 7708, 7712, 7820-7859 | 7718, 7722, 7726, 7729, 7741, 7743-7744, 7749-7750, 7757, 7760, 7764, 7860-7901 | 7770, 7774, 7778-7779, 7782, 7794, 7796-7797, 7803, 7806, 7811, 7818, 7902-7945 |
| 7:93106096_A_G | 7946-7997 | 7998-8051 | 8052-8107 |
| 7:93096736_CT_C rs201891812_REF | 8108-8127 | 8128-8143 | 8144-8159 |
| 7:93096736_CT_C rs201891812_SNP | 8115, 8119, 8121, 8160 | 8134, 8137 | 8153, 8156, 8161 |
| 7:93096863_C_T rs73216083_REF | 8162-8213 | 8214-8265 | 8266-8321 |
| 7:93096863_C_T rs73216083_SNP | 8162-8164, 8166, 8168, 8171, 8178, 8180, 8183, 8198, 8200, 8203, 8322-8361 | 8214-8217, 8220, 8223, 8231, 8234, 8249-8250, 8362-8403 | 8266-8269, 8271, 8273, 8276, 8283-8284, 8303, 8305-8306, 8404-8447 |
| 7:93096894_C_CTTTT rs542330750_REF | 8448-8466 | 8467-8486 | 8487-8507 |
| 7:93097829_G_A rs7798225_REF | 8508-8559 | 8560-8613 | 8614-8669 |
| 7:93097829_G_A rs7798225_SNP | 8508, 8513, 8515, 8523, 8527-8528, 8531, 8533, 8546, 8556, 8558-8559, 8670-8709 | 8560, 8567, 8575, 8579-8581, 8584, 8586, 8599, 8603, 8612-8613, 8710-8751 | 8614, 8621, 8629, 8633-8635, 8638, 8640, 8657-8658, 8665, 8669, 8752-8795 |
| 7:93099114_C_T rs10953089_REF | 8796-8831 | 8832-8861 | 8862-8896 |
| 7:93099114_C_T rsl0953089_SNP | 8805, 8808, 8811-8812, 8818, 8822-8823, 8827-8831, 8897-8912 | 8841, 8844-8845, 8849, 8853-8854, 8858-8861, 8913-8922 | 8869, 8872, 8875-8877, 8881, 8885-8886, 8892, 8895-8896, 8923-8934 |
| 7:93099215_G_A rs10953090_REF | 8935-8986 | 8987-9040 | 9041-9096 |
| 7:93099215_G_A rs10953090_SNP | 8938, 8941, 8945-8946, 8953, 8959, 8962, 8968-8969, 8973, 8975, 8981, 9097-9134 | 8990, 8993, 8997-8998, 9005, 9014, 9020-9021, 9025-9026, 9034, 9037, 9135-9176 | 9044, 9047, 9051, 9059, 9073, 9075-9076, 9080-9081, 9089, 9092-9093, 9177-9220 |
| 7:93099485_T_C rs10277338_REF | 9221-9266 | 9267-9302 | 9303-9346 |
| 7:93099485_T_C rs10277338_SNP | 9223, 9230, 9233, 9235, 9238, 9246, 9249, 9254-9255, 9260, 9262, 9264, 9347-9386 | 9274, 9276-9277, 9280, 9285, 9287, 9292-9293, 9297, 9299-9300, 9302, 9387-9426 | 9310, 9314, 9317, 9319, 9323-9324, 9326, 9334-9335, 9341-9342, 9344, 9427-9470 |
| 7:93100095_T_C rs71562815_REF | 9471-9494 | 9495-9518 | 9519-9542 |
| 7:93100095_T_C rs71562815_SNP | 9474, 9476-9478, 9480-9482, 9486-9487, 9489, 9491, 9494, 9543-9582 | 9498, 9500-9501, 9504-9505, 9509-9512, 9514-9515, 9518, 9583-9624 | 9522, 9524, 9527-9529, 9532-9536, 9539, 9542, 9625-9668 |
| 7:93100248_G_T rs12333976_REF | 9669-9712 | 9713-9744 | 9745-9778 |
| 7:93100248_G_T rs12333976_SNP | 9673-9674, 9679, 9685, 9694, 9700, 9779-9800 | 9715, 9718, 9724, 9730, 9736, 9741, 9801-9818 | 9747, 9756, 9762, 9768, 9773, 9777, 9819-9840 |
| 7:93100656_GT_G rs3839837_REF | 9841-9862 | 9863-9883 | 9884-9905 |
| 7:93100656_GT_G rs3839837_SNP | 9850, 9856-9857, 9860, 9862, 9906 | 9871, 9873, 9881, 9883, 9907 | 9889, 9892, 9894-9895, 9905, 9908 |
| 7:93104453_C_A rs10279499_REF | 5744, 5746, 5765, 5772, 5776, 5780, 5786, 5789, 9909-9952 | 5791, 5796, 5798, 5815, 5818, 5825, 5840, 5843, 9953-9998 | 5845, 5852, 5854, 5869, 5872, 5880, 5891, 5899, 9999-10046 |
| 7:93104453_C_A rs10279499_SNP | 5744, 5765, 5772, 5780, 9911, 9926, 9929-9930, 9932, 9934, 9938, 9948, 10047-10086 | 5791, 5796, 5815, 5818, 9955, 9970, 9974, 9977, 9981, 9991, 9993-9994, 10087-10128 | 5845, 5854, 5869, 5891, 10001, 10004, 10017, 10024, 10028, 10038, 10040, 10046, 10129-10172 |
| 7:93104662_G_A rs78564070_REF | 10173-10224 | 10225-10278 | 10279-10334 |
| 7:93104662_G_A rs78564070_SNP | 10175, 10178, 10184, 10188, 10196, 10201, 10206, 10208, 10213-10214, 10217, 10222, 10335-10374 | 10230, 10236, 10240, 10242, 10249, 10254, 10261, 10265, 10267-10268, 10271, 10276, 10375-10416 | 10284, 10290, 10296, 10303, 10308, 10315, 10319, 10321-10322, 10327, 10331-10332, 10417-10460 |
| 7:93105138_A_G rs6965201_REF | 6678-6679, 6681, 6688, 6692, 6696, 10461-10506 | 6710, 10507-10547 | 6714, 6722, 10548-10592 |
| 7:93105138_A_G rs6965201_SNP | 10466-10467, 10473, 10477, 10485-10486, 10488, 10490-10491, 10497, 10500, 10506, 10593-10632 | 10511-10512, 10518, 10520, 10523,10531-10533, 10535, 10543, 10633-10674 | 10552-10554, 10561, 10573-10575, 10577, 10579, 10586, 10588, 10675-10718 |
| 7:93105670_A_G rs6969691 REF | 10719-10761 | 10762-10801 | 10802-10844 |
| 7:93105670_A_G rs6969691_SNP | 10721, 10726, 10735, 10742-10743, 10746, 10752, 10845-10884 | 10764, 10768, 10776, 10786, 10791, 10797, 10885-10922 | 10804, 10816, 10818, 10826, 10829, 10834, 10840, 10923-10964 |
| 7:93106641_A_G rs2374627 REF | 10965-11016 | 11017-11069 | 11070-11125 |
| 7:93106641_A_G rs2374627_SNP | 10971, 10978, 10981-10982, 10996-10997, 10999, 11001, 11005-11007, 11011, 11126-11165 | 11023, 11030, 11032, 11034-11035, 11049-11050, 11054, 11058, 11060, 11064, 11166-11207 | 11073, 11077, 11084,11086, 11088-11089, 11096, 11104-11105, 11109, 11115-11116, 11208-11251 |
| 7:93106866_A_C rs12668221_REF | 11252-11303 | 11304-11357 | 11358-11413 |
| 7:93106866_A_C rs12668221_SNP | 11259, 11269, 11272, 11275, 11277, 11279, 11288, 11291-11292, 11298-11299, 11303, 11414-11453 | 11311, 11323, 11325, 11328, 11332, 11341, 11344-11345, 11351-11352, 11356-11357, 11454-11495 | 11365, 11372, 11379, 11381, 11384, 11388, 11400-11401, 11407-11408, 11412-11413, 11496-11539 |
| 7:93107482_A_C rs28673511 REF | 11540-11567 | 11568-11591 | 11592-11620 |
| 7:93107482_A_C rs28673511_SNP | 11540-11541, 11544, 11547, 11553-11554, 11557, 11561, 11621-11648 | 11568-11570, 11572, 11574, 11577, 11579, 11582,11649-11670 | 11592-11596, 11598, 11605, 11607, 11610, 11671-11694 |
| 7:93107663_T_C rs10229795_REF | 11695-11745 | 11746-11787 | 11788-11833 |
| 7:93107663_T_C rs10229795_SNP | 11699, 11708, 11713, 11718, 11721, 11724, 11726, 11729, 11737-11738, 11744, 11834-11873 | 11753, 11761, 11763, 11765, 11769, 11772, 11778, 11780, 11874-11915 | 11796, 11804, 11806, 11808, 11811, 11813, 11822-11823, 11825, 11829, 11916-11959 |
| 7:93107775_C_T rs7785869_REF | 11960-12003 | 12004-12047 | 12048-12091 |
| 7:93107775_C_T rs7785869_SNP | 11969, 11977, 11979, 11988, 11992, 11995, 12092-12129 | 12020, 12022, 12031, 12035, 12038, 12042, 12130-12167 | 12063, 12065, 12068, 12079, 12082, 12086, 12168-12205 |
| 7:93108136_C_T rs67885698_REF | 12206-12257 | 12258-12311 | 12312-12367 |
| 7:93108136_C_T rs67885698_SNP | 12206-12208, 12211, 12218-12219, 12226, 12233, 12238, 12245, 12252, 12255, 12368-12407 | 12258-12260, 12263, 12270, 12272, 12277, 12280, 12292, 12299, 12306, 12309, 12408-12449 | 12312-12314, 12324, 12326, 12331, 12345, 12347, 12354, 12361-12362, 12365, 12450-12493 |
| 7:93108745_C_G rs6957832 REF | 12494-12543 | 12544-12597 | 12598-12653 |
| 7:93108925_C_T rs12704664_REF | 12654-12665 | 12666-12679 | 12680-12695 |
| 7:93108925_C_T rs12704664_SNP | 12659-12662, 12664-12665, 12696-12707 | 12672-12674, 12676, 12678-12679, 12708-12727 | 12683, 12689-12690, 12692, 12694-12695, 12728-12755 |
| 7:93100828-7:93101327 (3′ UTR downstream to the stop codon) | 12756-13657 | 13658-14559 | 14560-15461 |
| 7:93099521-7:93100020 (3′ UTR upstream to the polyadenylation signal) | 15462-16255 | 16256-17073 | 17074-17909 |
| 7:93099413-7:93099913 (Intergenic) | 9221-9230, 9232-9264, 15463, 15465-15466, 15469, 15471, 15473-15478, 15480-15482, 15484-15486, 15488-15491, 15494-15500, 15502-15503, 15505-15508, 15512-15514, 15516-15521, 15523-15527, 15529-15530, 15532-15537, 15539, 15545-15546, 15548-15556, 15558-15561, 15563-15565, 15567-15574, 15576-15577, 15580- | 9267-9302, 16257-16258, 16260-16261, 16264, 16266, 16268-16273, 16275-16278, 16280-16282, 16284-16288, 16292-16298, 16300-16301, 16303-16306, 16310-16312, 16314-16319, 16321-16325, 16327-16328, 16330-16336, 16338, 16344-16345, 16347-16355, 16357-16360, 16362-16364, 16366-16373, 16375-16376, 16379-16381, | 9303-9346, 17075-17076, 17078-17079, 17082, 17084, 17087-17092, 17094-17097, 17099-17101, 17103-17108, 17112-17118, 17120-17121, 17123-17126, 17130-17132, 17134-17139, 17141-17145, 17147-17148, 17150-17156, 17158, 17164-17165, 17167-17176, 17178-17181, 17183-17185, 17187-17194, 17196-17197, 17200-17202, |
| | 15582, 15584-15586, 15588, 15591-15592, 15594-15601, 15603-15609, 15611-15615, 15617-15618, 15620-15631, 15635-15637, 15639-15641, 15643-15646, 15648-15656, 15658-15659, 15661-15663, 15672-15674, 15677, 15679, 15681-15682, 15684-15689, 15691-15693, 15695-15697, 15699-15701, 15703-15704, 15707, 15709-15711, 15713-15714, 15717-15723, 15725-15726, 15728-15731, 15734-15738, 15741-15742, 15744-15746, 15748-15753, 15755, 15757-15763, 15765-15766, 15768-15769, 15771, 15773-15781, 15784-15794, 15796-15798, 15800-15804, 15807-15811, 15813, 15817-15822, 15824-15825, 15827-15831, 15833-15837, 15840-15842, 15844-15849, 15851-15857, 15859-15865, 15867, 15869, 15871-15874, 15876-15880, | 16383-16385, 16387, 16390-16391, 16393-16400, 16402-16408, 16410-16414, 16416-16417, 16419-16430, 16434-16436, 16438-16440, 16442-16445, 16447-16455, 16457-16459, 16461-16463, 16472-16474, 16477, 16479, 16481-16482, 16484-16489, 16491-16493, 16495-16498, 16501-16503, 16505-16506, 16509, 16511-16513, 16515-16517, 16520-16526, 16528-16529, 16531-16534, 16537-16541, 16544-16545, 16547-16549, 16551-16556, 16558, 16560-16566, 16568-16569,16571-16572, 16574, 16576-16585, 16588-16598, 16600-16602, 16604-16608, 16611-16616, 16618, 16622-16627, 16629-16630, 16632-16636, 16638-16642, 16645-16647, 16649-16655, 16657-16663, 16665-16671, 16673, 16676-16679, 16681-16685, 16687-16692, | 17204-17206, 17208-17209, 17212-17213, 17215-17222, 17224-17230, 17232-17236, 17238-17239, 17241-17252, 17256-17258, 17260-17262, 17264-17267, 17269-17277, 17279-17281, 17283-17285, 17294-17296, 17299, 17301, 17304-17305, 17307-17312, 17314-17316, 17318-17321, 17324-17326, 17328-17329, 17332, 17334-17336, 17339-17341, 17344-17352, 17354-17355, 17357-17360, 17363-17367, 17370-17371, 17373-17375, 17377-17382, 17384, 17386-17392, 17394-17396, 17398-17399, 17401, 17403-17412, 17415-17425,17427-17429, 17431-17435, 17438-17443, 17445, 17449-17454, 17456-17457, 17459-17463, 17465-17469, 17472-17474, 17476-17482, 17484-17490, 17492-17498, 17500, 17503-17506, 17508-17512, 17514- |
| | 15882-15887, 15889-15894, 15897-15900, 15902, 15904-15908, 15910-15914, 15916-15928, 15930-15935, 15939-15947, 15949-15951, 15953-15957, 15960-15966, 15968-15977, 15979-15983, 15986-15992, 15997-15998, 16000-16003, 16005-16009, 16011, 16014-16020, 16023-16031, 16035-16036, 16038, 16041, 16045-16048, 16054-16055, 16059,16061-16063, 16065-16073, 16075-16078, 16080-16082, 16085-16088, 16090-16091, 16093, 16095-16096, 16098-16100, 16102-16103, 16106, 16108-16111, 16113-16116, 16118-16122, 16124-16127, 16129-16136, 16138-16145,16147-16148, 16150-16154, 16157, 16159-16161, 16164-16170, 16172-16177, 16181, 16184-16187, 16189-16198, 16200-16201, 16203, 16206-16215, 16218-16230, | 16694-16699, 16702-16705, 16707, 16709-16713, 16715-16719, 16721-16733, 16735-16740, 16744-16752, 16754-16756, 16758-16762, 16765-16766, 16768-16771, 16773-16782, 16784-16788, 16791-16798, 16803-16804, 16806-16809, 16811-16815, 16817, 16820-16826, 16829-16837, 16839, 16842-16843, 16845, 16848, 16852-16855, 16861-16863, 16867, 16869-16871, 16873-16881, 16883-16886, 16888-16890, 16893-16897, 16899-16900, 16902, 16904-16905, 16907-16908, 16910-16911, 16913-16916, 16919, 16921-16924, 16926-16929, 16931-16939, 16941-16948, 16950-16957, 16959-16960, 16962-16966, 16969, 16971-16973, 16976-16982, 16984-16989, 16993-16994, 16997-17000, 17002-17011, 17013-17014, 17016, 17019, 17021- | 17519, 17521-17526, 17529-17532, 17534, 17536-17540, 17542-17546, 17548-17560, 17562-17567, 17571-17579, 17581-17583, 17585-17589, 17592-17593, 17595-17599, 17601-17610, 17612-17616, 17619-17626, 17631-17633, 17635-17638, 17640-17644, 17646, 17649-17655, 17658-17666, 17668, 17671-17672, 17674-17675, 17678, 17682-17685, 17691, 17693-17695, 17699, 17701-17703, 17705-17714,17716-17719,17721-17723, 17726-17730, 17732-17733, 17735, 17737-17738, 17740-17741, 17743-17745, 17747-17750, 17754-17757, 17759-17762, 17764-17772, 17774-17781, 17783-17784, 17786-17790, 17792-17793, 17795-17800, 17803, 17805-17807, 17810-17816, 17818-17823, 17827-17828, 17831-17834,17836-17846, 17848- |
| | 16232, 16234, 16236-16240, 16242-16247, 16249-16250, 16252, 16255, 17910-18038 | 17029, 17032-17044, 17046, 17048, 17050-17055, 17057-17064, 17066-17068, 17070, 17073,18039-18182 | 17850, 17852, 17855-17864, 17867-17879, 17881, 17883, 17885-17890, 17892-17899, 17901-17903, 17905, 17907, 17909,18183-18322 |
| 7:93106106-7:93106605 (Intron 2) | 18323-19086 | 19087-19860 | 19861-20640 |

The indicated locations listed in column 1 of the Table 1 are based on gnomAD v3.1 database and UCSC Genome Browser assembly ID: hg38, Sequencing/Assembly provider ID: Genome Reference Consortium Human GRCh38.p12 (GCA_000001405.27). Assembly date: December 2013 initial release; December 2017 patch release 12.

The SNP details are indicated by the listed SNP ID Nos. (“rs numbers”), which are based on the NCBI 2018 database of Single Nucleotide Polymorphisms (dbSNP)). The indicated DNA mutations are associated with Transcript Consequence NM_017654 as obtained from NCBI RefSeq genes.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1: SAMD9 Correction Anaylsis

Guide sequences comprising 17-50 contiguous nucleotides containing nucleotides in the sequence set forth in any one of SEQ ID NOs: 1-20640 are screened for high on target activity using SpCas9 in HeLa cells. On target activity is determined by DNA capillary electrophoresis analysis.

### Example 2: Additional SAMD9 Editing Analysis

Germline gain of function mutations in sterile α motif domain-containing protein 9 (SAMD9) enhance its physiologic growth-inhibitory function and cause MIRAGE syndrome, which is characterized by growth restriction leading to bone marrow failure. To choose optimal RNA guide molecules for mutant allele elimination using a one-guide molecule strategy, which targets SNPs residing in the gene leading to protein truncation, or a two-guide molecule strategy deleting the full gene or 3′UTR of SAMD9, 14 different RNA guide molecules targeting nine (9) SNPs restricted to upstream of, downstream of, or within a coding region were screened for high on-target activity in HeLa and HSC cells (Table 2).

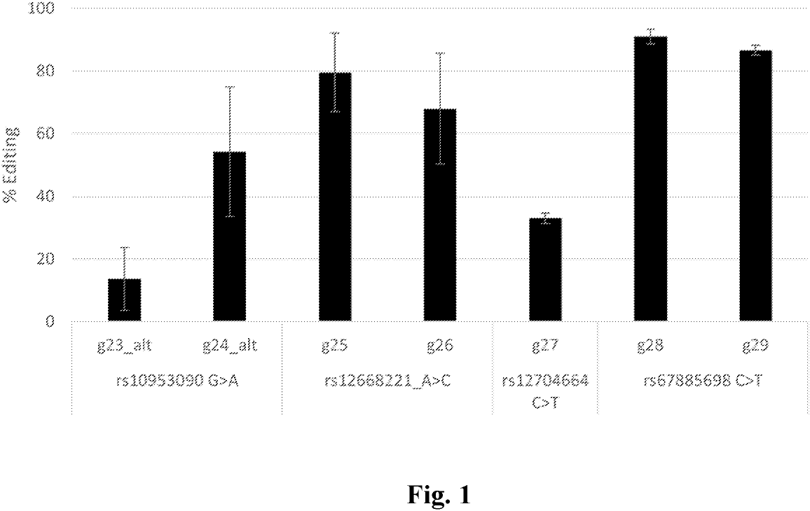

In HeLa cells, seven (7) different RNA guide moelcules targeting four (4) SNPs were screened by transfecting SpCas9 and a guide-expressing DNA molecule. Briefly, screening was performed in a 96-well format with an SpCas9 coding plasmid (64 ng) co-transfected with a DNA plasmid that expresses a RNA guide molecule (20 ng) using JetOPTIMUS® reagent (Polyplus). Cells were harvested 72 hours post DNA transfection. Cell lysis and genomic DNA extraction was performed in Quick extract (Lucigen) and endogenous genomic regions were amplified using specific primers to measure on-target activity by next-generation sequencing (NGS) (FIG. 1, Table 2).

Next, novel OMNI CRISPR nucleases with unique PAM requirements were tested for editing of upstream or coding region SNPs. These nucleases were tested in HeLa cells as described above. To this end, per each OMNI CRISPR nuclease, the corresponding OMNI-P2A-mCherry expression vector (pmOMNI, Table 4) was transfected into HeLa cells together with sgRNA designed to target specific location in the human genome (guide sequence portion, Table 2). At 72 hours cells were harvested, and half of the cells were used for quantification of transfection efficiency by FACS using mCherry fluorescence as marker. The rest of the cells were lysed, and their genomic DNA content was used in a PCR reaction which amplified the corresponding putative genomic targets. Amplicons were subjected to NGS and the resulting sequences were then used to calculate the percentage of editing events in each target site. Short insertions or deletions (indels) around the cut site are the typical outcome of repair of DNA ends following nuclease induced DNA cleavage. The calculation of % editing was therefore deduced from the fraction of indels containing sequences within each amplicon.

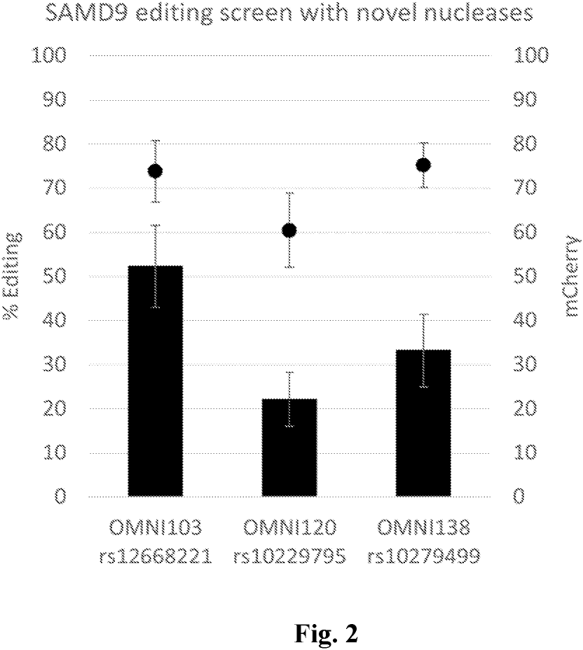

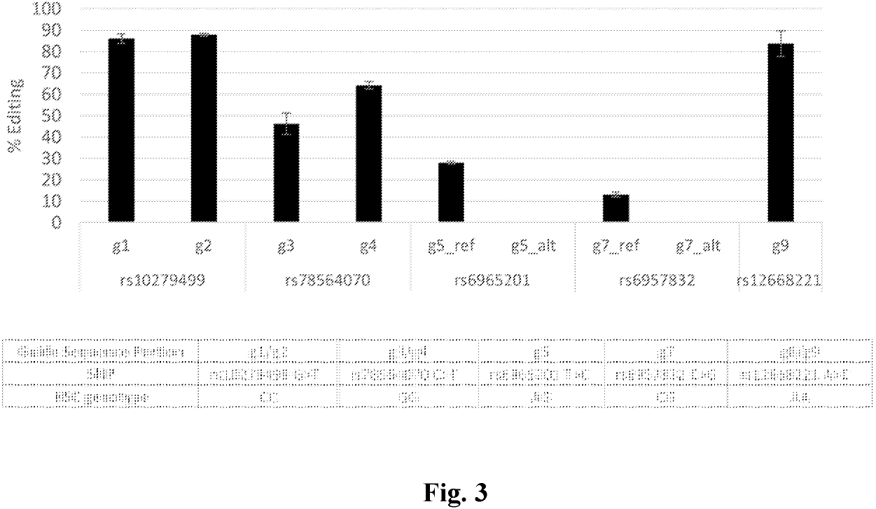

In addition, seven (7) additional, different RNA guide molecules targeting five (5) SNPs relevant to both excision and a one-guide strategy were screened in HSC cells. Briefly, 250 × 10³ HSC cells were mixed with preassembled RNPs composed of 105 pmole SpCas9 protein and 120 pmole of a sgRNA comprising a 20-nucleotide guide sequence portion, specified in Table 2, mixed with 100 pmole of electroporation enhancer (IDT-1075916) and electroporated using P3 primary cell 4D-nucleofector X Kit S (V4XP-3032, Lonza) by applying the DZ-100 program. A fraction of cells were harvested 72 hours post electroporation and genomic DNA was extracted to measure on-target activity by NGS. According to NGS analysis, all RNA guide molecules (FIG. 2, Table 2) depicted indel activity ranging from 10% -90%. Editing of g5 and g7 were allele specific (FIG. 3).

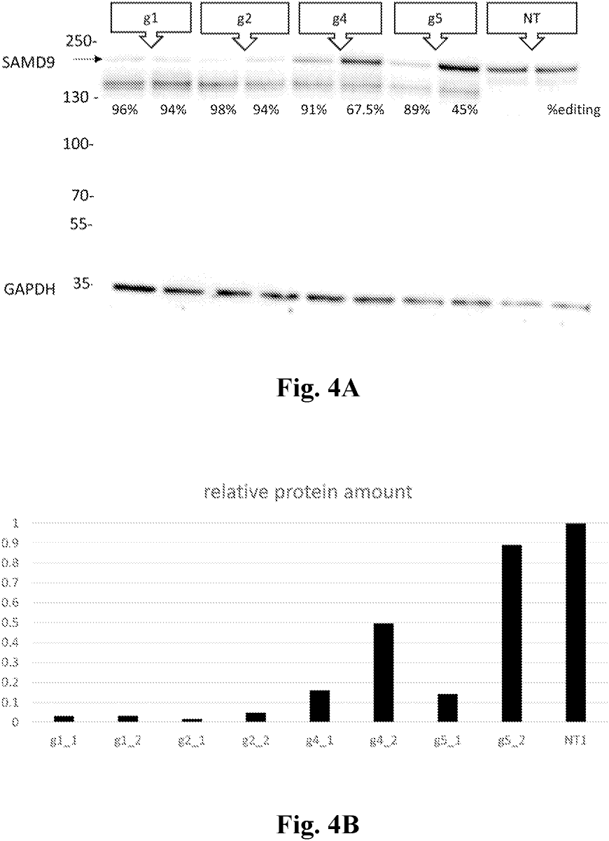

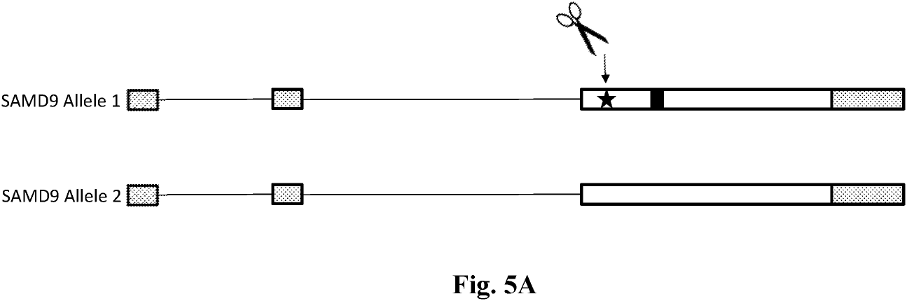

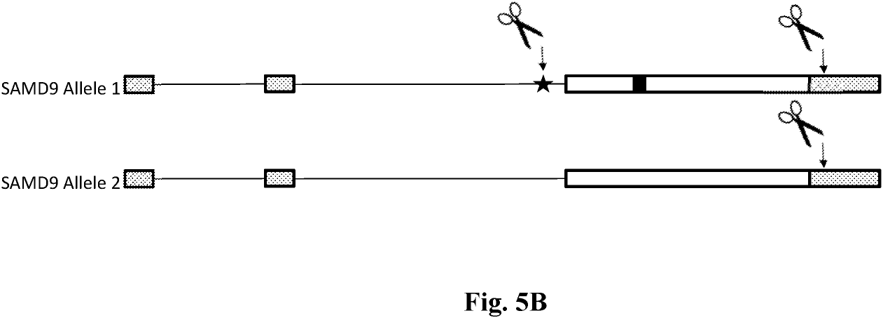

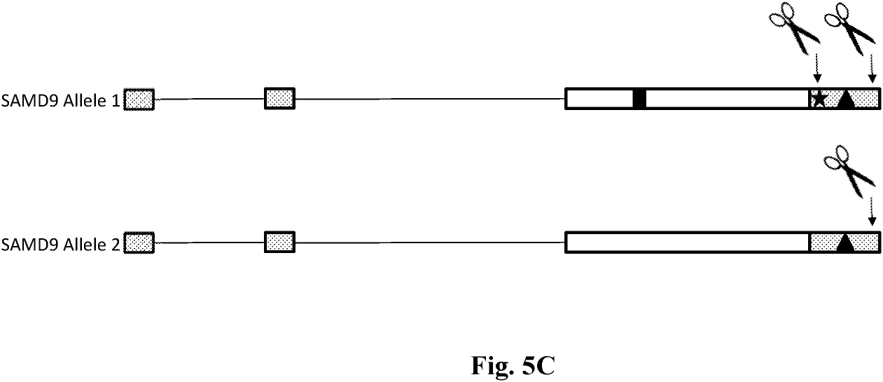

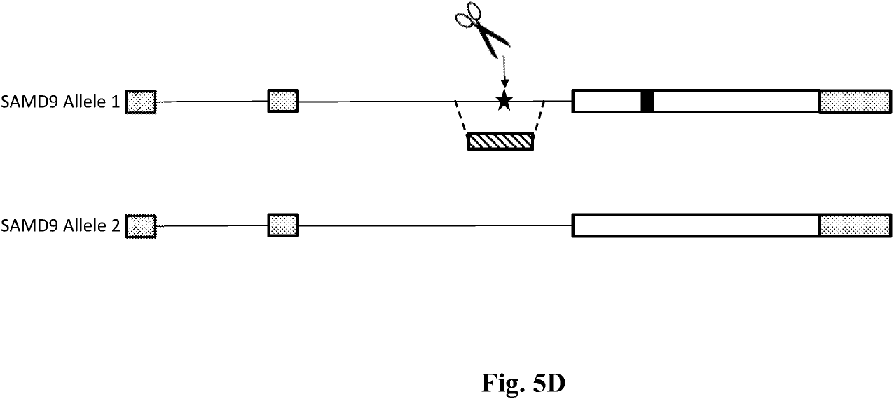

To test a one-guide molecule editing strategy of the mutant allele by targeting a SNP residing in the coding region of the gene (e.g. rs10279499, rs78564070 or rs6965201), which will lead to indels or frameshifts in the coding sequence and truncated protein, U2OS cells, homozygous to rs 10279499, rs78564070 and rs6965201 SNPs, were edited with an RNA guide molecule having one of four (4) different guide sequence portions. Briefly, 200 x 10³ U2OS cells were mixed with preassembled RNPs composed of 105 pmole SpCas9 protein and 120 pmole of a sgRNA comprising a 20-nucleotide guide sequence portion, specified in Table 2, mixed with 100 pmole of electroporation enhancer (IDT-1075916) and electroporated using SE primary cell 4D-nucleofector X Kit S (V4XC-1032, Lonza) by applying the DN-100 program. A fraction of cells were harvested 72 hours post electroporation, genomic DNA was extracted, and an on-target editing activity of 45-96% was measured by NGS. Cells were grown for an additional three (3) days and harvested for protein degradation. SAMD9 protein levels were detected with anti-SAMD9 antibody (ab180575, Abcam) and anti-GAPDH (60004-1-lg, Protein-tech) was used for normalization (FIGS. 3A, B). All guide sequence portions which depicted high editing efficiency resulted in significant decrease in SAMD9 levels (FIGS. 4A-4B) and can be used for a one-guide molecule strategy.

TABLE 2

20-nucleotide or 22-nucleotide long guide sequence portion sequences targeting SNPs located in SAMD9 region

| | | | | |
|---|---|---|---|---|
| Genomic region | SNP | Guide Sequence Portion (gRNA) Name | Guide Sequence Portion Sequence | PAM region |
| Coding region | rs10279499 | g1 | AUCUCUUGGGUCAUCCACAG (SEQ ID NO: 9913) | AGG |
| | | g2 | AUUUCUAUUACUGUCCUCUG (SEQ ID NO: 5746) | UGG |
| | | g30 | AUCUCUUGGGUCAUCCACAGAG (SEQ ID NO: 10003) | GACAGTAA |
| Coding region | rs78564070 | g3 | AGAGUAGAAAUCGUCUCAUU (SEQ ID NO: 10191) | UGG |
| | | g4 | AGAAAUCGUCUCAUUUGGUG (SEQ ID NO: 10189) | UGG |
| Coding region | rs6965201 | g5 | UAAUCUGGAAAUAAUCAUAU (SEQ ID NO: 10487) | UGG |
| Upstream | rs6957832 | g7 | GCAAGGCGGCAGCAAGCCUG (SEQ ID NO: 12525) | GGG |
| Upstream | rs12668221 | g9 | CAGAGUUGAGGUCUGUUUAA (SEQ ID NO: 11266) | AGG |
| | | g31 | GCCAAGACCCUUUAAACAGACC (SEQ ID NO: 11391) | TCAACTCT |
| Downstream | rs10953090 | g23_alt | AGAAGCAGAAAGUUGAAUGG (SEQ ID NO: 9108) | UGG |
| | | g24_alt | CAUAGAAGCAGAAAGUUGAA (SEQ ID NO: 9114) | UGG |
| Upstream | rs12668221 | g25 | AGAGUUGAGGUCUGUUUAAA (SEQ ID NO: 11261) | GGG |
| | | g26 | GAGGUCUGUUUAAAGGGUCU (SEQ ID NO: 11280) | UGG |
| Upstream | rs12704664 | g27 | CCCUGUCUGACAGCUUUGAA (SEQ ID NO: 12657) | AGG |
| Upstream | rs67885698 | g28 | UGGCGAGGCACGGCAAGGCA (SEQ ID NO: 12255) | CGG |
| | | g29 | GAGGCACGGCAAGGCACGGC (SEQ ID NO: 12233) | GGG |
| Upstream | rs10229795 | g32 | CUGAGUUUGUAUCUGCCACUAG (SEQ ID NO: 11808) | AAAACAAT |

TABLE 3

Novel nucleases sgRNA scaffold sequences, with each OMNI CRISPR nuclease also indicated with its PAM requirement

| | | |
|---|---|---|
| OMNI CRISPR Nuclease | PAM Sequence | sgRNA Scaffold Sequence |
| OMNI-103 (SEQ ID NO:20646) with g31 | NNRACT | GccaagacccUUUaaacagaccGUUUGAGAGUAGUGUAAgaaaUUACACUACAAGUUCAAAUAAAAAUUUAUUCAAAUCCAUUUGCUACAUUGUGUAGAAUUUAAAGAUCUGGCAACAGAUCUUUUUUU (SEQ ID NO: 20649) |
| OMNI-120 (SEQ ID NO: 20647 with g32 | NRRAC | CUgagUUUgUaUcUgccacUagGUUUGAGAGCCUUGUUAgaaaUAACAAGGCGAGUGCAAAUAAGGUUUAACCGAAUUCACCGUUUAUGGACCGCAUUGUGCGGAUUUUUU (SEQ ID NO: 20650) |
| OMNI-138 (SEQ ID NO: 20648) with g30 | NNNNGBNH | AUCUCUUGGGUCAUCCACAGAGGUUGUAGUUCCCUAAUUAUgaaaACAAUUAGGUUACUAUGAUAAGGUAGUAUACCGCAAAGCUCUAACGCCUCAUCUUAAUGAUGGGGCGUUAUCUUUUUU (SEQ ID NO: 20651) |

TABLE 4

OMNI CRISPR nuclease mammalian expression plasmid and elements

| | | |
|---|---|---|
| Plasmid Name | Purpose | Elements |
| pmOMNI | Expressing OMNI polypeptide in the mammalian system | CMV promoter - Kozak - SV40 NLS - OMNI ORF (human optimized) - HA - SV40 NLS - P2A - mCherry - bGH poly(A) signal |

TABLE 4

Annex

| | | |
|---|---|---|
| Element | SEQ ID NO of Amino Acid Sequence | SEQ ID NO of DNA sequence |
| HA Tag | SEQ ID NO: 20652 | SEQ ID NO: 20656 |
| NLS | SEQ ID NO: 20653 | SEQ ID NO: 20657 |
| P2A | SEQ ID NO: 20654 | SEQ ID NO: 20658 |
| mCherry | SEQ ID NO: 20655 | SEQ ID NO: 20659 |

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.

2. Anderson (1992) “Human gene therapy”, Science 256:808-13.

3. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.

4. Behr (1994) Gene transfer with synthetic cationic amphiphiles: Prospects for gene therapy”, Bioconjuage Chem 5:382-89.

5. Blaese (1995) “Vectors in cancer therapy: how will they deliver”, Cancer Gene Ther. 2:291-97.

6. Blaese et al. (1995) “T lympocyte-directed gene therapy for ADA-SCID: initial trial results after 4 years”, Science 270(5235):475-80.

7. Buchschacher and Panganiban (1992) “Human immunodeficiency virus vectors for inducible expression of foreign genes”, J. Virol. 66:2731-39.

8. Burstein et al. (2017) “New CRISPR-Cas systems from uncultivated microbes”, Nature 542:237-41.

9. Chang and Wilson (1987) “Modification of DNA ends can decrease end-j oining relative to homologous recombination in mammalian cells”, Proc. Natl. Acad. Sci. USA 84:4959-4963.

10. Chung et al. (2006) “Agrobacterium is not alone: gene transfer to plants by viruses and other bacteria”, Trends Plant Sci. 11(1):1-4.

11. Coelho et al. (2013) “Safety and efficacy of RNAi therapy for transthyretin amyloidosis” N. Engl. J. Med. 369, 819-829.

12. Collias and Beisel (2021) “CRISPR technologies and the search for the PAM-free nuclease”, Nature Communications 12, 555.

13. Crystal (1995) “Transfer of genes to humans: early lessons and obstacles to success”, Science 270(5235):404-10.

14. Davidsson et al. (2018) “SAMD9 and SAMD9L in inherited predispositon to ataxia, pancytopenia, and myeloid malignancies”, Leukemia 32:1106-1115.

15. Dillon (1993) “Regulation gene expression in gene therapy” Trends in Biotechnology 11(5):167-173.

16. Dranoff et al. (1997) “A phase I study of vaccination with autologous, irradiated melanoma cells engineered to secrete human granulocyte macrophage colony stimulating factor”, Hum. Gene Ther. 8(1): 111-23.

17. Dunbar et al. (1995) “Retrovirally marked CD34-enriched peripheral blood and bone marrow cells contribute to long-term engraftment after autologous transplantation”, Blood 85:3048-57.

18. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.

19. Gao and Huang (1995) “Cationic liposome-mediated gene transfer” Gene Ther. 2(10):710-22.

20. Haddada et al. (1995) “Gene Therapy Using Adenovirus Vectors”, in: The Molecular Repertoire of Adenoviruses III: Biology and Pathogenesis, ed. Doerfler and Böhm, pp. 297-306.

21. Han et al. (1995) “Ligand-directed retro-viral targeting of human breast cancer cells”, Proc Natl Acad Sci U.S.A. 92(21):9747-51.

22. Hu et al. (2018) “Evolved Cas9 variants with broad PAM compatibility and high DNA specificity”, Nature 556(7699):57-63.

23. Inaba et al. (1992) “Generation of large numbers of dendritic cells from mouse bone marrow cultures supplemented with granulocyte/macrophage colony-stimulating factor”, J Exp Med. 176(6):1693-702.

24. Jinek et al. (2012) “A programmable dual-RNA-guided DNA endonuclease in adaptive bacterial immunity,” Science 337(6096):816-21.

25. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.

26. Judge et al. (2006) “Design of noninflammatory synthetic siRNA mediating potent gene silencing in vivo”, Mol Ther. 13(3):494-505.

27. Kohn et al. (1995) “Engraftment of gene-modified umbilical cord blood cells in neonates with adnosine deaminase deficiency”, Nature Medicine 1:1017-23.

28. Kremer and Perricaudet (1995) “Adenovirus and adeno-associated virus mediated gene transfer”, Br. Med. Bull. 51(1):31-44.

29. Macdiarmid et al. (2009) “Sequential treatment of drug-resistant tumors with targeted minicells containing siRNA or a cytotoxic drug”, Nat Biotehcnol. 27(7):643-51.

30. Malech et al. (1997) “Prolonged production of NADPH oxidase-corrected granulocyes after gene therapy of chronic granulomatous disease”, PNAS 94(22):12133-38.

31. Miller et al. (1991) “Construction and properties of retrovirus packaging cells based on gibbon ape leukemia virus”, J Virol. 65(5):2220-24.

32. Miller (1992) “Human gene therapy comes of age”, Nature 357:455-60.

33. Mitani and Caskey (1993) “Delivering therapeutic genes - matching approach and application”, Trends in Biotechnology 11(5): 162-66.

34. Nabel and Felgner (1993) “Direct gene transfer for immunotherapy and immunization”, Trends in Biotechnology 11(5):211-15.

35. Nishimasu et al. (2018) “Engineered CRISPR-Cas9 nuclease with expanded targeting space”, Science 361(6408):1259-1262.

36. Narumi et al. (2016) “SAMD9 mutations cause a novel multisystem disorder, MIRAGE syndrome, and are associated with loss of chromosome 7”, Nature Genetics 48:792-797.

37. Remy et al. (1994) “Gene Transfer with a Series of Lipphilic DNA-Binding Molecules”, Bioconjugate Chem. 5(6):647-54.

38. Sentmanat et al. (2018) “A Survey of Validation Strategies for CRISPR-Cas9 Editing”, Scientific Reports 8:888, doi:10.1038/s41598-018-19441-8.

39. Sommerfelt et al. (1990) “Localization of the receptor gene for type D simian retroviruses on human chromosome 19”, J. Virol. 64(12):6214-20.

40. Van Brunt (1988) “Molecular framing: transgenic animals as bioactors” Biotechnology 6:1149-54.

41. Vigne et al. (1995) “Third-generation adenovectors for gene therapy”, Restorative Neurology and Neuroscience 8(1,2): 35-36.

42. Walton et al. (2020) “Unconstrained genome targeting with near-PAMless engineered CRISPR-Cas9 variants”, Science 368(6488):290-296.

43. Weissman and Kariko (2015) “mRNA:Fulfilling the promise of gene therapy”, Molecular Therapy (9):1416-7.

44. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.

45. Yu et al. (1994) “Progress towards gene therapy for HIV infection”, Gene Ther. 1(1):13-26.

46. Zetsche et al. (2015) “Cpf1 is a single RNA-guided endonuclease of a class 2 CRIPSR-Cas system” Cell 163(3):759-71.

47. Zuris et al. (2015) “Cationic lipid-mediated delivery of proteins enables efficient protein based genome editing in vitro and in vivo” Nat Biotechnol. 33(1):73-80.

1. A method for modifying in a cell a mutant allele of the sterile alpha motif domain containing 9 (SAMD9) gene having a mutation associated with MIRAGE syndrome, the method comprising
introducing to the cell a composition comprising:
at least one CRISPR nuclease or a sequence encoding a CRISPR nuclease; and
a first RNA molecule comprising a guide sequence portion having 17-50 nucleotides or a nucleotide sequence encoding the same,
wherein a complex of the CRISPR nuclease and the first RNA molecule affects a double strand break in the mutant allele of the SAMD9 gene.

2. The method of claim 1, wherein the first RNA molecule targets the CRISPR nuclease to a SNP position of the mutant allele,
wherein the SNP position is any one of rs10279499, rs201891812, rs73216083, rs542330750, rs7798225, rs10953089, rs10953090, rs10277338, rs71562815, rs12333976, rs3839837, rs78564070, rs6965201, rs6969691, rs2374627, rs12668221, rs28673511, rs10229795, rs7785869, rs67885698, rs6957832 and rs12704664;
wherein the guide sequence portion of the first RNA molecule comprises 17-50 contiguous nucleotides containing nucleotides in the sequence set forth in any one of SEQ ID NO: 9913, SEQ ID NOs: 1-9912, or SEQ ID NOs: 9914-20640 that targets a SNP position of the mutant allele;
wherein the SNP position is in an exon of the SAMD9 mutant allele; and/or
wherein the SNP position contains a heterozygous SNP.

3-6. (canceled)

7. The method of claim 1, wherein the first RNA molecule targets the CRISPR nuclease to the mutation associated with MIRAGE syndrome,
wherein the mutation associated with MIRAGE syndrome is any one of 7:93101391 C G, 7:93101435 GA, 7:93101512 C T, 7:93101540 C A, 7:93101615 T C, 7:93101761 AGTT A, 7:93101900 G A, 7:93102220 C T, 7:93102221 G A, 7:93102242 G T, 7:93102259 G A, 7:93102368 T C, 7:93102514 G A, 7:93102593 G A, 7:93102692 C G, 7:93102717 G T, 7:93103150 AT GA, 7:93103151 T C, 7:93103153 C T, 7:93103178 C T, 7:93103253 C A, 7:93103393 C T, 7:93103588 C CT, 7:93103598 T A, 7:93103628 G A, 7:93103679 C T, 7:93103765 G A, 7:93103772 G T, 7:93103793 C T, 7:93103851 G T, 7:93103900 A G, 7:93103916 T C, 7:93103933 G T, 7:93103938 GT G, 7:93104044 C T, 7:93104176 A G, 7:93104296 ATT A, 7:93104449 T A, 7:93104694 TAC GCA, 7:93104695 AC GA, 7:93104722 C T, 7:93105043 A G, 7:93105103 A G, 7:93105122 G A, 7:93105295 A G, 7:93105436 C T, 7:93105485 T G, 7:93105638 G A, 7:93105781 C T, 7:93105965 CT C, 7:93106002 GT G, 7:93106003 T A, and 7:93106096 A G; and/or
wherein the guide sequence portion of the first RNA molecule comprises 17-50 contiguous nucleotides containing nucleotides in the sequence set forth in any one of SEQ ID NOs: 1-20640 that targets a mutation associated with MIRAGE syndrome.

8-9. (canceled)

10. The method of claim 7, further comprising introduction of a donor molecule that encodes a synthetic splice site.

11. The method of claim 1, further comprising introducing to the cell a second RNA molecule comprising a guide sequence portion having 17-50 nucleotides or a nucleotide sequence encoding the same, wherein a complex of the second RNA molecule and a CRISPR nuclease affects a second double strand break in the SAMD9 gene.

12. The method of claim 11, wherein the guide sequence portion of the second RNA molecule comprises 17-50 contiguous nucleotides containing nucleotides in the sequence set forth in any one of SEQ ID NOs: 1-20640 other than the sequence of the first RNA molecule.

13. The method of claim 11, wherein the second RNA molecule comprises a non-discriminatory guide portion that targets both functional and mutated SAMD9 alleles.

14. The method of claim 11, wherein the second RNA molecule comprises a non-discriminatory guide portion that targets any one of a SAMD9 untranslated region (UTR), an intergenic region upstream of SAMD9, an intergenic region downstream of SAMD9, or Intron 2 of SAMD9;
wherein the second RNA molecule comprises a non-discriminatory guide portion that targets a sequence that is located within a genomic range selected from any one of 7:93100828 to 7:93101327, 7:93099521 to 7:93100020, 7:93099413 to 7:93099913, or 7:93106106 to 7:93106605; and/or
wherein the second RNA molecule comprises a non-discriminatory guide portion that targets a sequence that is located up to 500 base pairs from the sequence targeted by the first RNA molecule.

15-16. (canceled)

17. The method of claim 11, wherein a portion of an exon is excised from the mutant allele of the SAMD9 gene.

18. The method of claim 11, wherein the first RNA molecule targets a SNP position in the 3′ UTR of the mutated allele, and the second RNA molecule comprises a non-discriminatory guide portion that targets downstream of a polyadenylation signal sequence that is common to both a functional allele and the mutant allele of the SAMD9 gene; or
wherein the first RNA molecule targets a SNP position downstream of a polyadenylation signal of the mutated allele, and the second RNA molecule comprises a non-discriminatory guide portion that targets a sequence upstream of a polyadenylation signal that is common to both a functional allele and the mutant allele of the SAMD9 gene.

19. (canceled)

20. The method of claim 18, wherein the polyadenylation signal is excised from the mutant allele of the SAMD9 gene.

21. A modified cell obtained by the method of claim 1.

22. A first RNA molecule comprising a guide sequence portion having 17-50 contiguous nucleotides containing nucleotides in the sequence set forth in any one of SEQ ID NOs: 1-20640.

23. A composition comprising the first RNA molecule of claim 22 and at least one CRISPR nuclease.

24. The composition of claim 23, further comprising a second RNA molecule comprising a guide sequence portion having 17-50 contiguous nucleotides, wherein the second RNA molecule targets a SAMD9 allele, and wherein the guide sequence portion of the second RNA molecule is a different sequence from the sequence of the guide sequence portion of the first RNA molecule.

25. The composition of claim 24, wherein the guide sequence portion of the second RNA molecule comprises 17-50 contiguous nucleotides containing nucleotides in the sequence set forth in any one of SEQ ID NOs: 1-20640 other than the sequence of the first RNA molecule.

26. A method for inactivating a mutant SAMD9 allele in a cell, the method comprising delivering to the cell the composition of claim 22.

27. A method for treating MIRAGE syndrome, the method comprising delivering to a cell of a subject having MIRAGE syndrome the composition of claim 22.

28. (canceled)

29. A medicament comprising the composition of claim 23 for use in inactivating a mutant SAMD9 allele in a cell, wherein the medicament is administered by delivering to the cell the composition of claim 23.

30. (canceled)

31. A medicament comprising the composition of claim 23 for use in treating ameliorating or preventing MIRAGE syndrome, wherein the medicament is administered by delivering to a cell of a subject having or at risk of having MIRAGE syndrome the composition of claim 23.
